(19) United States
(12) Patent Application Publication
(10) Pub. No.: US 2026/0144653 A1
(43) Pub. Date: May 28, 2026

(54) PROSTHETIC ARM WITH HYBRID BRAIN-COMPUTER INTERFACE

(71) Applicant: Northern Kentucky University, Highland Heights, KY (US)

(72) Inventors: Mahdi Yazdanpour, Cincinnati, OH (US); Yangyang Tao, Highland Heights, KY (US); Vu Tran, Highland Heights, KY (US)

(73) Assignee: Northern Kentucky University, Highland Heights, KY (US)

(21) Appl. No.: 19/398,647

(22) Filed: Nov. 24, 2025

Related U.S. Application Data
(60) Provisional application No. 63/724,952, filed on Nov. 26, 2024.

(51) Int. Cl. A61F 2/72; A61F 2/54; A61F 2/58; A61F 2/70; G06F 3/01

(52) U.S. Cl. CPC A61F 2/72; A61F 2/585; A61F 2002/543; A61F 2002/546; A61F 2002/587; A61F 2002/704; G06F 3/015

(57) ABSTRACT

A brain-controlled robotic prosthetic arm integrates a hybrid neural network model, combining Convolutional Neural Networks and Recurrent Neural Networks for advanced electroencephalography signal processing. The system enables users to control the prosthetic arm using brain signals, with supplementary integration of manual operation. The modular design allows for user-attachable extension parts, customizable configurations, and 3D-printed components tailored to the user's anatomy. Each finger is actuated by an independent micro linear motor, providing precise control and lifelike movements. The prosthetic arm incorporates a sensory feedback system, with flexible material on the fingertips embedded with sensors that detect tactile forces, temperature, and pressure. This feedback is delivered in real time to enhance the user's perception of interaction with their environment. The hybrid neural network utilizes transfer learning and Generative Adversarial Networks for data augmentation, addressing challenges in electroencephalography signal variability and data scarcity to improve classification accuracy.

### REFERENCE TO RELATED APPLICATION

The present application claims priority to U.S. Provisional Patent Application Ser. No. 63/724,952, filed 26 Nov. 2024, titled Prosthetic Arm with Hybrid Brain-Computer Interface, the disclosure of which is expressly incorporated herein by reference.

### STATEMENT REGARDING FEDERALLY SPONSORED RESEARCH OR DEVELOPMENT

This invention was made with government support under Northern Kentucky University (NKU) Internal Grant #4001678, External Subaward #3048115221-23-135, awarded by Kentucky Network for Innovation & Commercialization (KYNETIC) Cycle 2, funded by the National Institute of Health (NIH) Research Evaluation and Commercialization Hub; and under NKU Internal Grant #4001894, External Subaward #ULRF25-0088A-CS-07, awarded by Mid-South REACH, Funded by the National Institute of Health (NIH) Research Evaluation and Commercialization Hub. The government has certain rights in the invention.

### FIELD OF THE INVENTION

This invention relates to the fields of prosthetics, robotics, human-machine interfaces, artificial intelligence, and neural engineering. It specifically addresses brain-controlled prosthetic limbs that integrate sensory feedback, modular architecture, and a hybrid Convolutional Neural Network (CNN)+Recurrent Neural Network (RNN) model for processing electroencephalography (EEG) signals. The invention aims to create a more affordable, robust, natural, and functional upper-limb prosthetic by combining non-invasive neural signal interpretation with real-time sensory feedback. It is designed for applications in medical rehabilitation, assistive technology, and human augmentation, providing a significant improvement over existing prosthetic solutions in terms of user adaptability, control, and sensory integration.

The prosthetic limb itself addresses the need for advanced upper-limb prosthetic devices that provide natural control over individual finger movements, realistic sensory feedback, and aesthetically pleasing design. The invention incorporates modular design principles, allowing for customizable configurations to accommodate different levels of amputation and user preferences. It features lightweight actuation mechanisms and real-time sensory feedback systems to enhance daily usability, comfort, and functionality. Additionally, the prosthetic arm includes manual operation capabilities via a switch for basic control, integration with EEG-based brain-computer interfaces (BCIs) and EMG-based systems for intuitive control, and a microcomputer that serves as the control unit, managing signals from multiple input sources and optimizing the system's power consumption. In one embodiment, the prosthetic limb incorporates a multi-material fused structure printed on a single manufacturing cycle. This structure integrates rigid and flexible regions without the need for discrete fasteners, bearings, or springs.

The control system for the prosthetic arm addresses the use of transfer learning to improve CNN+RNN model generalization for EEG-based BCIs, tackling challenges such as inter-subject variability, noisy data, and limited data availability. It focuses on combining CNNs for spatial feature extraction and RNNs for capturing temporal dependencies, with transfer learning adapting models across different subjects and tasks. The invention provides a novel approach to applying transfer learning to CNN+RNN models for EEG signal processing, achieving higher classification accuracy for tasks like motor imagery while mitigating issues related to non-stationary EEG signals. While the control system is discussed herein primarily in connection with prosthetic control, the system can be extended to BCI applications in neurorehabilitation, assistive robotics, and other instances where EEG classification is required across various users and environments.

The control system further includes a method and system for enhancing EEG datasets using adversarial networks for upsampling, particularly in the context of hybrid CNN+RNN architectures. Generative Adversarial Networks (GANs) are used to generate synthetic EEG data, augmenting small and imbalanced datasets common in BCI applications. Specifically, the GAN approach involves training a generator to produce realistic EEG signals that mimic the original data's distribution, while a discriminator evaluates the authenticity of generated samples. Through adversarial training, the generator continuously improves, creating a larger dataset with diverse samples that retain the inherent characteristics of real EEG data. This upsampling technique helps overcome limited training data and class imbalances in EEG datasets, enhancing the robustness and accuracy of CNN+RNN classification models used in motor imagery and other BCI-related tasks.

### BACKGROUND OF THE INVENTION

Traditional upper-limb prosthetics, especially those utilizing electromyography (EMG)-based control systems, face several limitations in terms of cost, accuracy, and adaptability. EMG-based prosthetic arms typically require multiple surface sensors placed on the skin to detect electrical muscle activity, which can lead to inconsistent signal quality due to factors such as sensor displacement, sweat, skin conditions, or muscle fatigue. These limitations can compromise the precision and reliability of control, particularly during prolonged use. Moreover, some EMG-based systems are invasive or semi-invasive, adding to their cost and complexity, and potentially limiting accessibility for a wider population.

In contrast, brain-computer interface (BCI) technologies offer a promising alternative by using EEG signals to control robotic devices based on brain activity. EEG signals, which reflect electrical brain activity, are widely used in BCIs to decode brain signals for direct communication between humans and external devices. This technology has gained significant attention due to its applications in medical rehabilitation, neurofeedback systems, and assistive robotics. These technologies are entirely non-invasive, measuring electrical activity from the scalp, thus eliminating the need for invasive procedures. However, there is currently no commercially available EEG-based prosthetic arm that can utilize BCI technology for real-time and accurate control. Most existing solutions rely on muscle-based control methods, which suffer from limitations such as muscle fatigue, signal variability, and the need for precise sensor placement, making them less suitable for continuous use and less appealing to a broader user base. Also, the complexity of EEG data—characterized by its non-stationary nature, noise, and variability across individuals—poses significant challenges in extracting meaningful features for classification and control.

Several key research problems and challenges persist in this area, including:
EEG Signal Complexity: EEG data is inherently noisy, non-stationary, and exhibits high variability across different subjects and within the same subject over time. These factors make it difficult to extract meaningful features and classify the signals accurately.
Spatial and Temporal Dependencies: The data contains both spatial correlations across electrode locations and temporal variations over time, necessitating sophisticated modeling techniques to capture both dimensions effectively.
Data Scarcity: The availability of high-quality, labeled EEG datasets is often limited, posing challenges for training deep learning models that require large amounts of data.
Real-Time Performance: BCIs designed for assistive technologies, such as robotic control, must process EEG data in real-time, balancing the need for high accuracy with low latency.
Interpretability: Deep learning models are frequently regarded as “black boxes,” making it challenging to understand how specific features of EEG signals contribute to the final classification. This issue is especially relevant in clinical and rehabilitation contexts, where interpretability is crucial.

With respect to the prosthetics themselves, upper-limb prosthetics have traditionally faced significant limitations in terms of control, adaptability, and user experience. Many existing prosthetic devices lack the ability to provide natural, fine-grained control of individual finger movements, which restricts their effectiveness for tasks requiring dexterity, such as typing, handling delicate objects, or using tools. Additionally, the absence of sensory feedback prevents users from perceiving contact forces, temperature, or texture, making it difficult to interact naturally with their environment.

Current state-of-the-art prosthetic arms often rely on mechanical linkages or basic motorized systems that do not offer a full range of motion or sufficient degrees of freedom. These solutions can be bulky and heavy, resulting in user fatigue, discomfort, and limited wear time. The need for frequent maintenance and complex customization can further hinder the widespread adoption of advanced prosthetics.

Another challenge in robotic prosthetics is the lack of modularity, which limits customization and makes it difficult to accommodate different amputation levels or evolving user needs. Many prosthetic arms are designed as rigid systems with fixed components, offering limited flexibility for upgrades, repairs, or adaptations. There is a pressing need for a more versatile approach that allows for easy replacement or reconfiguration of individual parts, such as fingers, wrist joints, or sensory modules.

Sensory feedback is crucial for enhancing the functionality and user experience of robotic prosthetics. Without the ability to sense tactile forces, temperature, or grip pressure, users cannot adjust their grip strength or respond to environmental conditions, leading to risks such as dropping objects or damaging items. Integrating sensory feedback technologies into a prosthetic arm can significantly improve the sense of embodiment and control, allowing users to perform everyday tasks with greater ease and confidence.

### SUMMARY

The present invention addresses the issues discussed above and other needs by introducing a modular, brain-controlled robotic prosthetic arm equipped with a hybrid neural network model that combines CNNs for spatial feature extraction and RNNs for temporal analysis of EEG signals. The prosthetic arm includes a microcomputer that processes real-time signals from various input sources, including EEG-based BCIs, EMG-based control systems, and manual operation modes. The system integrates these multiple control modalities, providing flexibility and adaptability for different user preferences and needs.

The robotic prosthetic arm of a first embodiment features a modular design that allows for customizable components, such as fingers, thumb, and wrist modules. Each finger is equipped with independent actuators to enable precise and natural control over individual movements. The prosthetic arm of a second embodiment incorporates a multi-material fused structure printed in a single manufacturing cycle, hereinafter referred to as the FusionLimb configuration. This structure integrates rigid and flexible regions without the need for discrete fasteners, bearings, or springs. The arm of the first or second embodiment integrates a sensory feedback system with sensors that provide tactile, force, and temperature feedback, which is relayed to the user in real time to facilitate a more natural interaction with objects and the environment. The modular architecture of the arm supports easy attachment, removal, and customization, accommodating different amputation levels and evolving user needs. Lightweight actuators are used to deliver a realistic range of motion while minimizing power consumption, making the prosthetic suitable for daily use. The invention also includes a manual operation mode, allowing basic finger movements to be controlled via a switch, as well as integration capabilities for EEG-based BCIs and EMG-based systems, offering intuitive and natural control.

A microcomputer housed in the forearm section of the prosthetic arm serves as the control unit, processing signals from the manual switch, sensory feedback system, and external interfaces (EEG/EMG), while managing power distribution to optimize battery life. This invention addresses key challenges in existing prosthetics by providing a versatile, functional, and user-friendly solution that enhances adaptability, sensory integration, and control precision. The hybrid neural network leverages transfer learning techniques and synthetic data generation using GANs to overcome challenges associated with EEG signal variability and data scarcity. The transfer learning process fine-tunes the model to each individual user, enabling the system to achieve high classification accuracy in decoding motor imagery tasks. GANs are used to augment the training datasets, creating synthetic EEG data that improves the generalization capability of the neural network across different users and task variations.

To address challenges in the control system, and the inherent complexity of EEG signal processing in particular, this invention provides an advanced method and system for EEG signal classification in BCI, using a hybrid neural network architecture that integrates CNN and RNN. The CNN component is designed for extracting spatial features from EEG signals, while the RNN component models the temporal dependencies inherent in sequential data. This combination enables the system to effectively capture both the spatial and temporal characteristics of EEG signals, significantly improving classification accuracy in BCI applications, such as motor imagery tasks, neurorehabilitation, and assistive robotics.

One aspect of the invention is the use of transfer learning to enhance model generalization across different subjects and tasks. By fine-tuning a pre-trained CNN on new datasets, the invention adapts the model to the specific characteristics of the target data, even when high-quality labeled EEG datasets are limited. This approach leverages knowledge from related tasks to improve performance, addressing the data scarcity issue that is prevalent in EEG signal processing.

To further overcome the limitations of small and imbalanced datasets, the invention employs GAN for data augmentation. The GAN generates synthetic EEG signals that mimic the distribution of real data, expanding the training dataset and increasing its diversity. This augmentation technique not only enhances the robustness of the model but also helps balance class distributions, which is critical for achieving high classification accuracy.

The invention also addresses the need for real-time processing and interpretability in BCI systems. The hybrid CNN-RNN model is optimized for deployment on portable devices, such as Raspberry Pi or NVIDIA Jetson Nano, allowing for real-time EEG signal processing and control of external devices. Additionally, techniques like attention mechanisms and saliency maps can be integrated to make the model's decision-making process more interpretable, providing insights into the specific features of EEG signals that influence classification outcomes.

In one embodiment, the present invention is a prosthetic arm apparatus comprising a plurality of interconnected sections arranged adjacent to each other, the sections including a hand section connected to a wrist connector, the wrist connector connected to a wrist section, the wrist section connected to a forearm section; wherein the hand section includes a palm section connected to the wrist connector; a knuckles joint connected to the palm section; a plurality of fingers connected to the knuckles joint; a thumb connected to the palm section; and a plurality of actuators; wherein each of the plurality of fingers includes a proximal phalanx connected to the knuckle joint, a middle phalanx connected to the proximal phalanx, and a distal phalanx connected to the middle phalanx, where each of the proximal, middle, and distal phalanges is covered by a flexible thermoplastic material, and wherein the distal phalanx includes an internal cavity having at least one sensor positioned within the internal cavity; and a line extending from the distal phalanx, through the middle phalanx, through the proximal phalanx, and operatively connected to one of the plurality of actuators in the hand section; wherein the thumb includes a thenar connected to the palm section, a thumb proximal phalanx connected to the thenar, and a thumb distal phalanx connected to the thumb proximal phalanx; a line extending from the thumb distal phalanx, through the thumb proximal phalanx, through the thenar, and operatively connected to one of the plurality of actuators in the hand section; wherein the forearm section includes a microcomputer and a battery, wherein the plurality of actuators are in wired or wireless communication with the microcomputer and the battery. In some embodiments, activation of the actuators by the microcomputer retracts or extends the lines, transitioning the hand between an open state and a closed state.

In prosthetic arm system, the forearm section can be connected to an elbow section, which is further connected to an upper arm extension. Also, a switch can be used which permits for manual opening and closing of the hand. Even further, the prosthetic arm can include a control system including at least one non-transitory computer readable storage medium having computer program instructions stored thereon; and at least one processor configured to execute the computer program instructions causing the processor to perform the following operations: (a) receive electroencephalography (EEG) signals from a user of a neurodetection device, (b) segment the received EEG signals by a collection duration to produce segmented EEG signals, (c) preprocess the segmented EEG signals by providing bandpass filtering, notch filtering, and transformer-based denoising to produce preprocessed EEG signals; (d) decode, using a machine-learning system, the preprocessed EEG signals to detect imagined spatial motion of the user, where the imagined spatial motion corresponds to desired motion commands of the prosthetic arm; (e) execute, using one or more of the plurality of actuators, detected imagined spatial motion of the user; wherein the machine-learning system is a hybrid system including a first neural network and a second neural network, wherein the received EEG signals are input to the first neural network, wherein output of the first neural network is input to the second neural network, and wherein output of the second neural network is classification and identification of imagined spatial motion. Also, the neurodetection device can include a plurality of EEG signal-detecting electrodes worn on the user's head.

In addition, the prosthetic arm can be brain-controlled by use of: a neurodetection device configured to acquire electroencephalography (EEG) signals from a user; a preprocessing module configured to filter and denoise the EEG signals; a hybrid neural network decoder configured to classify neural intent from the EEG signals; and a control unit housed in the prosthetic arm and configured to activate one or more actuators of the prosthetic arm in response to the classified neural intent.

In another embodiment, the present invention is a prosthetic arm system with a prosthetic arm apparatus having: a plurality of interconnected sections arranged adjacent to each other, the sections including a hand section connected to a wrist connector, the wrist connector connected to a wrist section, the wrist section connected to a forearm section; wherein the hand section includes: a palm section connected to the wrist connector; a plurality of fingers connected to the palm section; a thumb connected to the palm section; and a plurality of actuators, each finger and the thumb having at least one actuator associated therewith; wherein the plurality of fingers are produced as a single continuous printed component comprising a rigid load-bearing material, each finger having a joint flex connector which acts as a flexible hinge material and provides a mechanical connection that allows bending at a finger joint while maintaining structural integrity, each finger further having a restorative band which provides elastic restoring force to the finger, where after the associated actuator bends the finger, the restorative band pulls it back to the initial straight position once actuation is released; wherein the thumb is produced as a single continuous printed component comprising a rigid load-bearing material, the thumb having a joint flex connector which acts as a flexible hinge material and provides a mechanical connection that allows bending at a thumb joint while maintaining structural integrity, the thumb further having a restorative band which provides elastic restoring force to the thumb, where after the associated actuator bends the thumb, the restorative band pulls it back to the initial straight position once actuation is released; and wherein the forearm section includes a microcomputer and a battery, wherein the plurality of actuators are in wired or wireless communication with the microcomputer and the battery. Each finger may include a knuckle which connects the fingers to the palm. Also, the proximal, middle, and distal phalanges of the fingers are covered by a flexible thermoplastic material, and each of the distal phalanx includes an internal cavity having at least one sensor positioned within the internal cavity.

The disclosures in paras [0027] and [0028] apply to the prosthetic arm system embodiment disclosed in para [0029] as well.

In another embodiment, the present invention is a system for controlling a prosthetic arm including a plurality of actuators, the system comprising at least one non-transitory computer readable storage medium having computer program instructions stored thereon; and at least one processor configured to execute the computer program instructions causing the processor to perform the following operations: receive electroencephalography (EEG) signals from a user of a neurodetection device; segment the received EEG signals by a collection duration; decode, using a machine-learning system, the segmented EEG signals to detect imagined spatial motion of the user, where the imagined spatial motion corresponding to desired motion commands for the prosthetic arm; execute, using one or more of the plurality of actuators, detected imagined spatial motion of the user; wherein the machine-learning system is a hybrid system including a first neural network and a second neural network, wherein the received EEG signals are input to the first neural network, wherein output of the first neural network is input to the second neural network, and wherein output of the second neural network is classification and identification of imagined spatial motion. In further embodiments, the neurodetection device includes a plurality of EEG signal-detecting electrodes worn on the user's head.

Further, any prosthetic arm apparatus disclosed herein can be used with system for controlling a prosthetic arm. Also any prosthetic arm apparatus disclosed herein can include a switch which permits for manual opening and closing of the hand.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 is a back view of the robotic prosthetic arm, including the hand of the first embodiment, wrist and forearm sections and two attachable extension parts: the elbow section and upper arm section.

FIG. 2 is an exploded front perspective view of the robotic arm.

FIG. 3 is an isometric front view of the robotic prosthetic arm.

FIG. 4 is an isometric back view of the robotic prosthetic arm.

FIG. 5 is a volumetric shaded isometric front view of the robotic prosthetic arm with two attachable extension parts.

FIG. 6 is a volumetric shaded isometric back view of the robotic prosthetic arm with two attachable extension parts.

FIG. 7 is a front view of the hand, illustrating the fingers, palm and wrist.

FIG. 8 is a volumetric shaded front view of the hand, illustrating the fingers and soft fingerprints.

FIG. 9 is a back view of the hand, illustrating the fingers, dorsum, and the location of the manual switch.

FIG. 10 is a volumetric shaded back view of the hand also showing the location of the manual switch.

FIG. 11 is an exploded back perspective view of the hand.

FIG. 12 is a back view showing the placement of micro linear actuators inside the hand, detailing the locations and configuration for each finger.

FIG. 13 is an isometric side view of the thumb components, highlighting the rotary mechanism and its connection to the thenar.

FIG. 14 is an isometric side view of the thumb, showing the spring return mechanism that assists with thumb repositioning.

FIG. 15 is an isometric side view of the component that drives the thumb using a line, showing the routing and attachment points including the thumb bearing and rod.

FIG. 16 is a side bottom perspective view of the thumb housing, illustrating the connection between the line and the thumb actuator housing.

FIG. 17 is a bottom perspective view of the finger actuators, showing the arrangement and linkages used for driving individual finger movements.

FIG. 18 is a top view showing the internal structure of the main actuator housing and knuckle joints within the hand.

FIG. 19A is a back view of a detachable finger and soft fingerprint.

FIG. 19B is a front perspective view of a detachable finger and soft fingerprint and also showing the internal cavity.

FIG. 20A is a back view of a finger, illustrating the location of the phalanx joint springs.

FIG. 20B is a front view of a finger with a soft fingerprint, illustrating the location of the finger bearing.

FIG. 21A is a back view of a finger and portion of the hand, showing the location of a micro linear actuator.

FIG. 21B is a front view of a finger and portion of the hand, showing the location of a micro linear actuator.

FIG. 22 is a top perspective view of the arm, showing the placement of the wrist servo motor, microcomputer, and battery.

FIG. 23 is a schematic illustrating the overall design of the Hybrid Brain-Computer Interface (Hybrid-BCI).

FIG. 24 shows an example of an EEG signal captured from the brain, highlighting the signal's characteristics and variability.

FIG. 25 is a schematic illustrating the architecture of the CNN+RNN model, including the layers for CNN and RNN, the input data structure, and the model's output.

FIG. 26 is a schematic depicting the transfer learning process used to fine-tune the pre-trained CNN, detailing the two main steps: feature extraction and fine-tuning.

FIG. 27 is a schematic illustrating the preprocessing steps for the EEG signals, including the use of a GAN to augment the dataset by upsampling.

FIG. 28 is an illustrated flow chart showing the GAN architecture, demonstrating how the generator and discriminator networks are trained adversarially to improve the quality of synthetic EEG data.

FIG. 29 is a schematic illustrating the I/O interface, detailing the acquisition of EEG data and the preparation steps for training the model and performing classification tasks.

FIG. 30 is a back view of the FusionLimb hand of the second embodiment, illustrating the fingers and dorsum.

FIG. 31 is a volumetric shaded back view of the FusionLimb hand.

FIG. 32 is a front view of the FusionLimb hand, illustrating the fingers, palm and wrist

FIG. 33 is a volumetric shaded front view of the FusionLimb hand, illustrating the fingers and soft fingerprints.

FIG. 34 is a back view of the FusionLimb prosthetic arm including the hand, wrist and forearm sections and two attachable extension parts: the elbow section and upper arm section.

FIG. 35 is a volumetric shaded isometric back view of the robotic prosthetic arm with two attachable extension parts.

FIG. 36 is a front view of the FusionLimb prosthetic arm including the hand, wrist and forearm sections and two attachable extension parts.

FIG. 37 is a volumetric shaded isometric front view of the FusionLimb prosthetic arm with two attachable extension parts.

FIG. 38A shows the finger in the straight position with two flexible parts, a Joint Flex Connector and a Restorative Band.

FIG. 38B is a shaded view of the finger in the straight position with two flexible parts, a Joint Flex Connector and a Restorative Band.

FIG. 39A shows the finger in the bent position with two flexible parts, Joint Flex Connector and Restorative Band.

FIG. 39B is a shaded view of the finger in the bent position with two flexible parts, Joint Flex Connector and Restorative Band.

### DETAILED DESCRIPTION OF THE PREFERRED EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter. As used herein, the term “about,” when referring to a value or to an amount is meant to encompass variations of ±10% of the most precise digit in the value or amount (e.g., “about 1” refers to 0.9 to 1.1, “about 1.1” refers to 1.09 to 1.11, etc.). The term “substantially,” when modifying a term associated with a number, has the same meaning as “about” (e.g., “substantially perpendicular” to an element means an orientation with ±10% of 90 degrees with respect to that element).

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

For the purposes of orientation with respect to the drawings, the “front” of the prosthetic arm is the direction of the palm, the “back” is the direction opposite the back, the “top” is the direction of the thumb, the “bottom” is the direction opposite the thumb, and the “side” refers to the lateral edges of the arm, perpendicular to the top and bottom. It should be understood that the prosthetic arm may be positioned in a variety of orientations when worn, so the terms “front,” “back,” “top,” “bottom,” and “side” are intended only for ease of understanding the drawings and referring to the relative positioning of components and are not intended to be limiting.

The following reference numbers are used in the specification and drawings: 1—hand section, 2—wrist, 3—forearm section, 4—elbow extension, 5—upper arm extension, 6—forearm cover, 7—wrist connector, 8—thumb proximal phalanx, 9—palm, 10—knuckles joint, 11—thumb distal phalanx, 12—flex fingerprint, 13—finger joint, 14—finger proximal phalanx, 15—flex middle phalanx, 16—finger distal phalanx, 17—flex fingerprint, 18—dorsum, 19—wrist servo motor, 20—first actuator pointer finger, 21—second actuator middle finger, 22—third actuator ring finger, 23—fourth actuator little finger, 24—fifth actuator thumb, 25—thumb actuator housing, 26—main actuator housing, 27—thenar, 28—spring, 29—thumb bearing, 30—line, 31—rod, 32—sensors, 33—sensors, 34—microcomputer, 35—battery, 36—restorative band, 37—joint flex connector, 38—internal cavity, 39—switch, 50—pointer or index finger, 51—middle finger, 52—ring finger, 53—little or pinky finger, 54—thumb, 61—FusionLimb hand, 100—prosthetic arm first embodiment, and 110—prosthetic arm second embodiment which includes the FusionLimb hand.

The invention provides a first (100) and a second (110) embodiment of a brain-controlled robotic prosthetic arm that combines advanced neural signal processing with a modular mechanical design. The hybrid neural network model, consisting of CNNs and RNNs, processes EEG signals to decode motor imagery tasks, allowing the user to control the prosthetic limb in real time. CNNs capture spatial features from the EEG data, detecting patterns across multiple channels, while RNNs analyze the temporal dependencies to enhance the accuracy of classification.

In one embodiment, the prosthetic arm is directly controlled by the user's brain activity. Electroencephalography (EEG) signals obtained from the user are segmented, preprocessed (including filtering and transformer-based denoising), and decoded by a hybrid neural network comprising a convolutional neural network (CNN) and a recurrent neural network (RNN). The resulting neural-decoding output is transmitted to the microcomputer of the prosthetic arm, which activates one or more micro linear actuators to perform the corresponding finger, thumb, wrist, elbow, or arm movements. Thus, the system forms a fully integrated brain-controlled prosthetic arm that enables the user to execute motor actions using only neural intent.

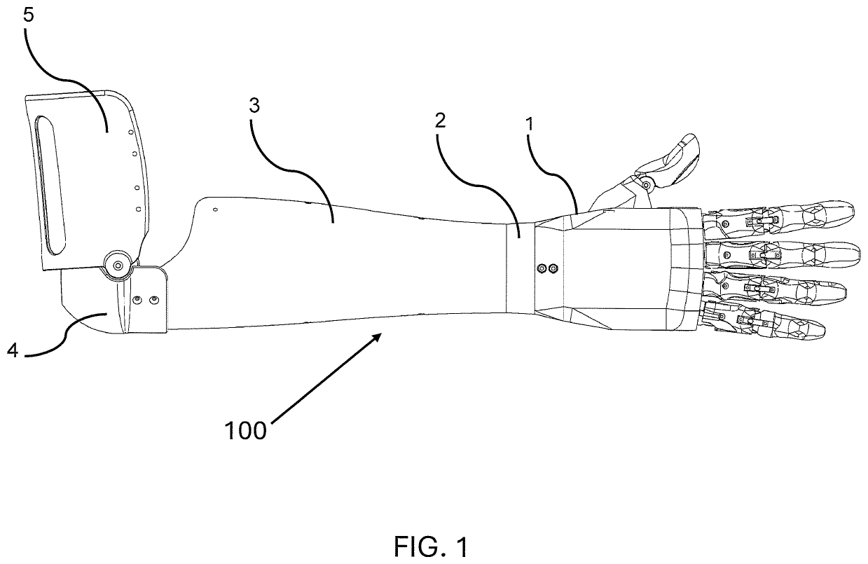

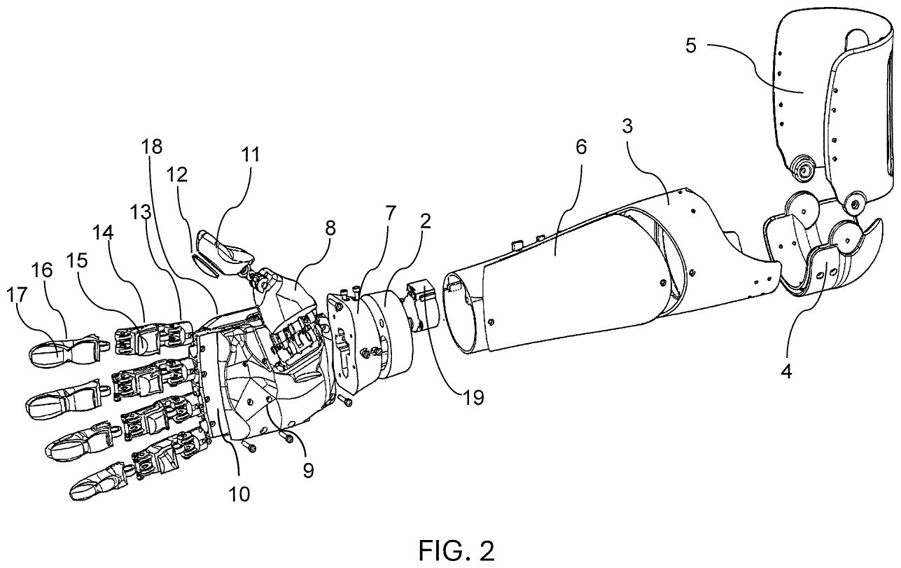

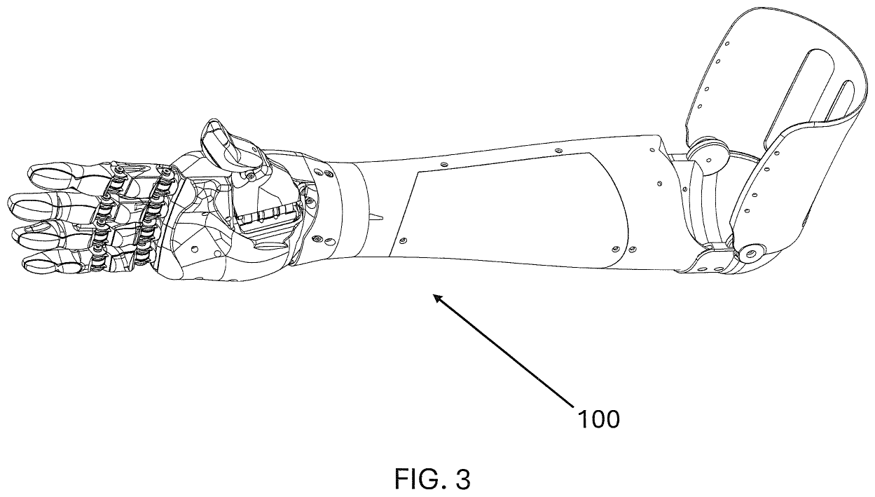

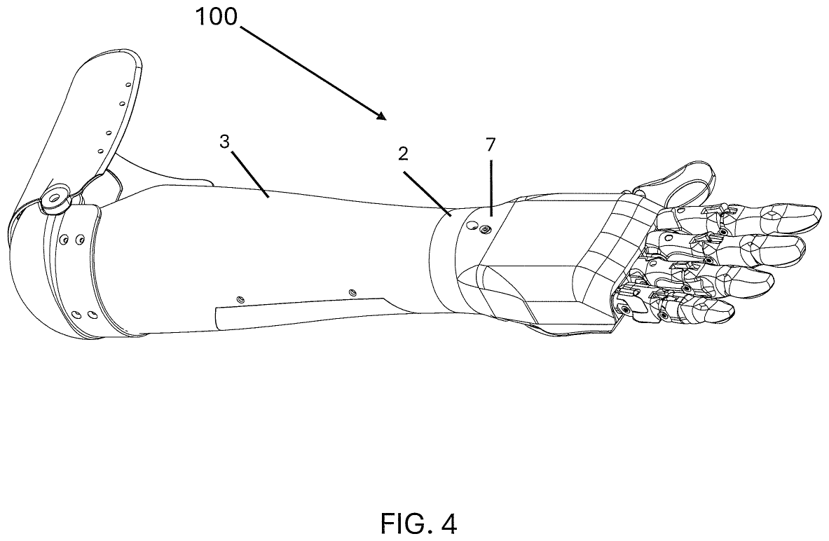

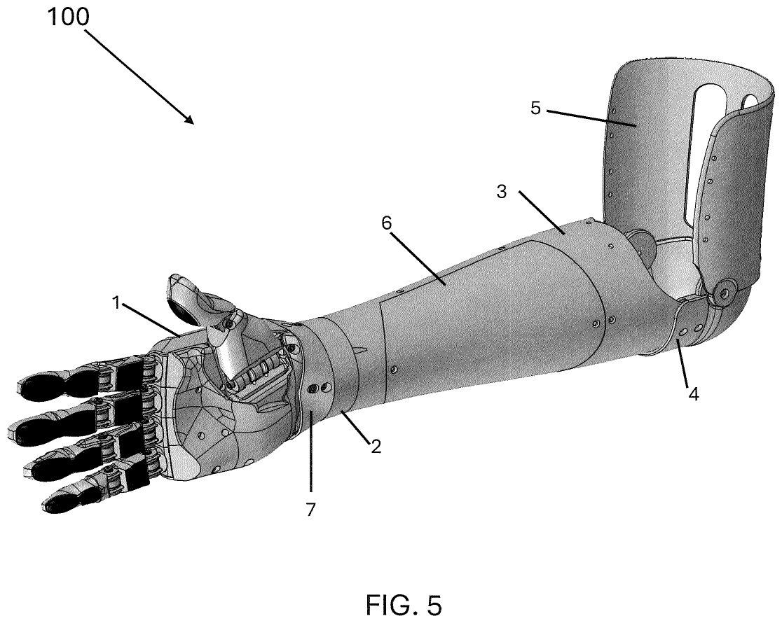

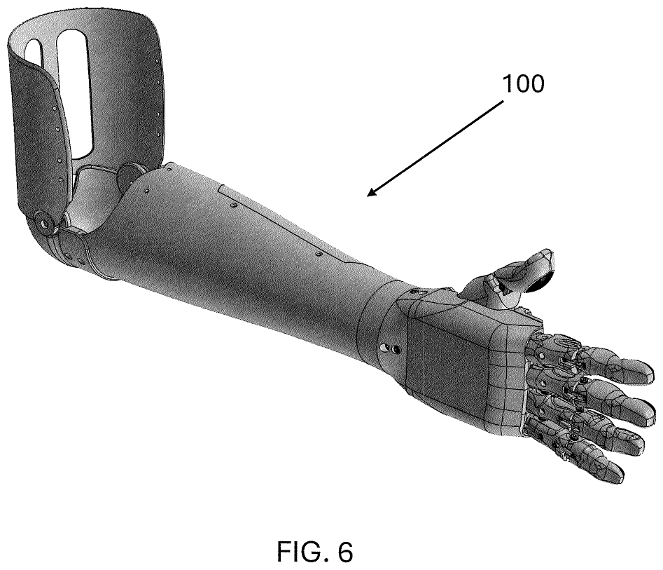

The robotic prosthetic arm of the first embodiment (100) features a modular design that allows for customized configurations to suit different user needs. The arm consists of two main sections: the hand section (1) and the forearm section (3), which connect at the wrist (2) and wrist connector (7) as shown in FIGS. 1 through 6. The modular architecture enhances versatility, ease of assembly, maintenance, and customization for various amputation levels. Components of the prosthetic arm include independent finger actuation, a sensory feedback system, efficient actuation mechanisms, and a control unit.

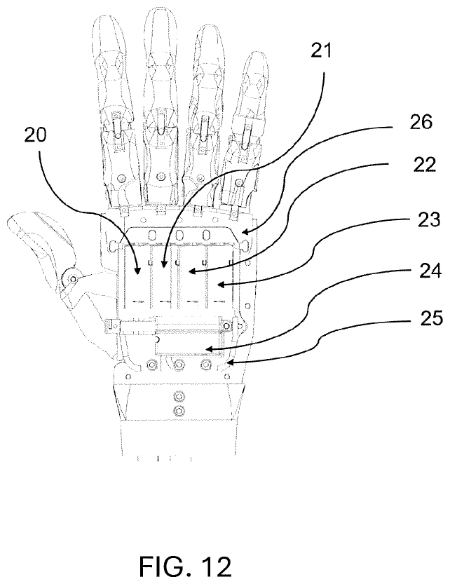

Each finger is operated by an individual micro linear actuator, namely, first actuator (20), second actuator (21), third actuator (22), and fourth actuator (23), and the thumb is operated by fifth actuator (24), as most easily seen in FIG. 12, which drives movement through a system of lines. The lines are preferably formed of a strong, lightweight material such as, for example, polytetrafluoroethylene (PTFE) or nylon. These micro linear motors provide precise control over finger movements, enabling fine motor tasks and realistic dexterity. The compact size and efficiency of the micro linear motors contribute to the arm's lightweight nature, allowing for a more comfortable user experience and reducing power consumption.

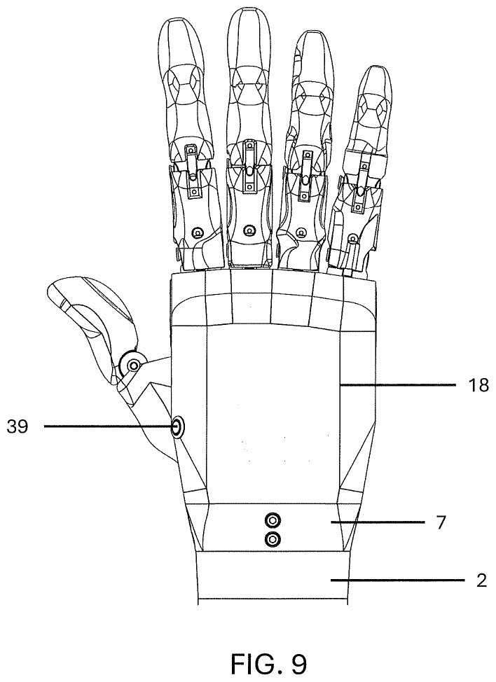

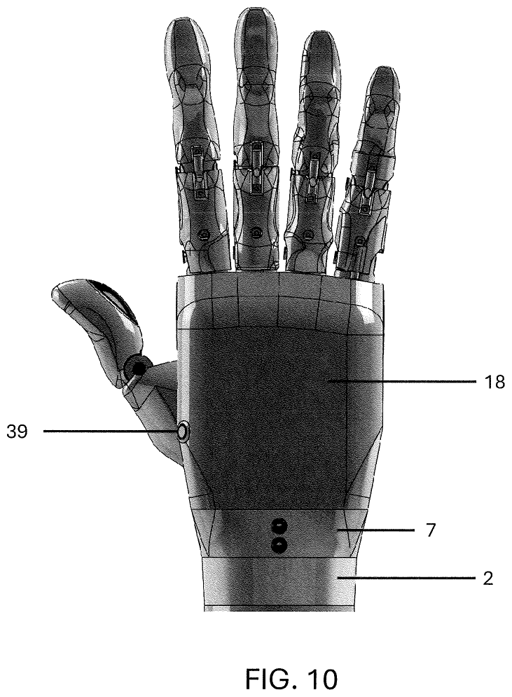

Manual Operation: The prosthetic arm allows for manual operation using a switch (39) to open or close the fingers. The user can engage this feature to control the hand without the need for external signals, providing an alternative method for performing basic tasks and improving accessibility. In some embodiments, the switch (39) is located on the left side of the dorsum (back) of the hand as is shown in FIGS. 9 and 10. Activation of the switch (39) sends a signal to the programmed control unit, which then triggers the micro linear motors to actuate each finger and the thumb, moving them into a closed position. Pressing the switch (39) again reverses the process, returning the fingers to an open position

Integration with EEG and EMG Interfaces: The prosthetic arm is designed for seamless integration with various human-machine interfaces, including EEG-based BCIs and electromyography (EMG)-based control systems. These interfaces allow the user to operate the arm using neural signals or muscle activity, enabling more natural and intuitive control of the prosthetic. Machine learning-based signal processing technique, such as those described below, and embedded connectivity ports allow compatibility with external devices. The modular design of the arm supports the attachment of external sensors and electrodes, while lightweight materials and flexible internal wiring maintain functionality and adaptability.

The arm is equipped with a microcomputer (34) housed in the forearm that acts as the control unit, processing signals from manual switches, EEG or EMG interfaces, and embedded sensors (32, 33). The microcomputer (34) coordinates finger movements, interprets sensory feedback data, and manages power consumption to optimize the life of the battery (35).

Weight and Balance Considerations: While the prosthetic arm aims to be lightweight, reducing the weight below that of a natural human arm is not always ideal for all users. Amputees may be accustomed to the absence of extra weight on their limb. Therefore, the design focuses not only on reducing the overall weight but also on optimizing the distribution of weight. Careful attention is given to positioning the center of mass (CoM) close to the elbow to minimize fatigue and discomfort, ensuring that the arm feels natural during extended use. In some embodiments, portions of the prosthetic arm are made by additive manufacturing using carbon fiber-reinforced PA6 (Nylon 6) filament or other strong and lightweight material. Thinner layers are used near the hand to reduce weight, while thicker layers near the elbow provide stability and shift the CoM rearward. This weight distribution, along with small weights near the elbow, reduces strain and ensures a natural feeling during extended use, providing both comfort and functionality

Battery Life and Power Efficiency: The prosthetic arm is designed to function for an entire day on a single battery charge. All components, including the battery, electronics, and mechanical systems, are optimized for power efficiency through several design features. Lightweight, low-power motors minimize energy consumption, while advanced control algorithms ensure precise operation and reduce unnecessary power usage. These algorithms process sensory feedback (e.g., from EEG/EMG or sensors) in real-time with minimal computational overhead, further conserving energy and reducing strain on the microcomputer. This ensures that a portable battery can provide sufficient power for a full day of continuous use without compromising performance. The power management system monitors and adjusts the energy consumption of each component to maximize battery life.

Biocompatibility and Skin Contact: Since the prosthetic arm is in prolonged contact with the skin, the biocompatibility of materials is essential. The materials selected for use in the arm are skin-friendly and non-toxic, chosen to prevent irritation or adverse reactions during extended wear. The use of these biocompatible materials enhances the comfort and long-term wearability of the prosthetic, ensuring it is suitable for daily use. In some embodiments, components in direct contact with the user, such as the casing of the forearm section (3), elbow section (4), and upper arm extension (5) are made of resin-based biocompatible material. In certain embodiments, the fingerprints are made of soft and flexible thermoplastic polyurethane (TPU), which is also a biocompatible material. All materials, as well as preferred substitutes, are non-toxic and non-irritating to skin, reducing the risk of adverse reactions during extended wear and promoting user comfort.

The palm of the prosthetic arm includes the following components: Cover, back of hand: A protective outer layer that shields the back of the hand, ensuring durability and safeguarding the internal components. Palm (9): The main section where the wiring and sensors are integrated. It provides structural support and houses the internal systems necessary for controlling the actuators and processing sensory feedback. Main actuator housing (26) is a central hub that contains all actuators (20-24) and connects all the wiring from the sensors and actuators (20-24) within the hand, enabling coordinated control and seamless signal transmission.

Fingers and Thumb Construction: The hand section (1) comprises the fingers, thumb, and palm (9), and dorsum (18) each engineered for realistic and precise movements, closely mimicking the structure and function of a natural human hand. Each finger includes multiple parts, including distal phalanx (16), proximal phalanx (14), flex middle phalanx (15), finger joint (13) and flexible fingerprint (17). Similarly, the thumb includes distal phalanx (11), a proximal phalanx (8), thenar (27) and flexible fingerprint (12). These components cooperatively allow the bionic hand to replicate natural dexterity and perform various tasks.

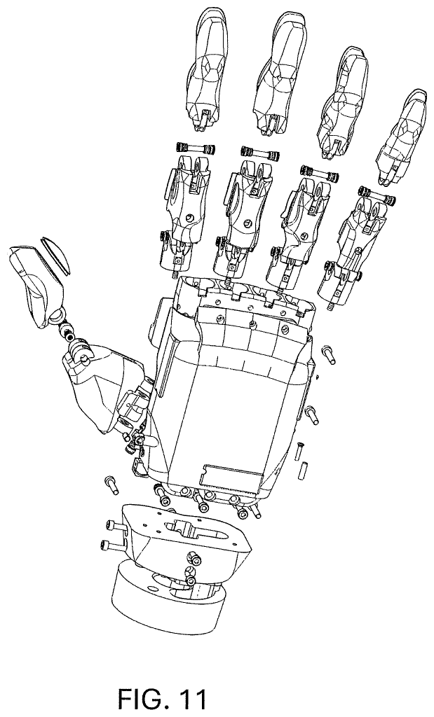

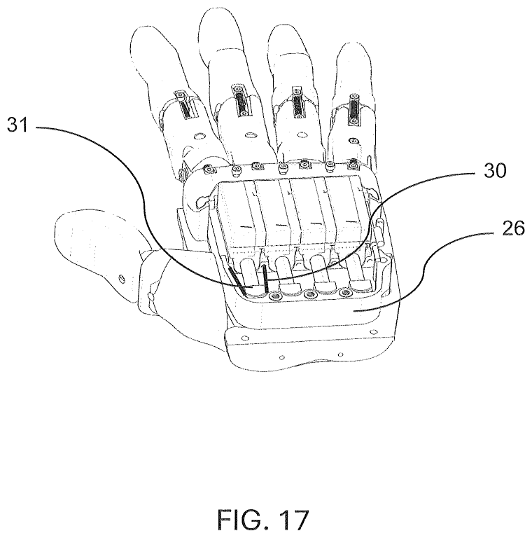

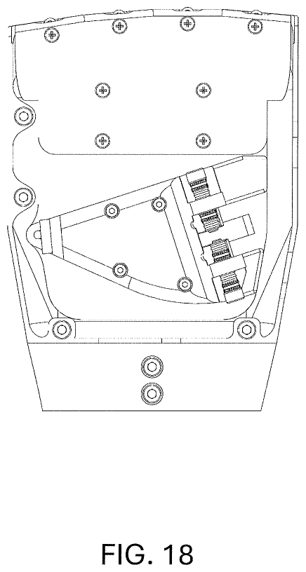

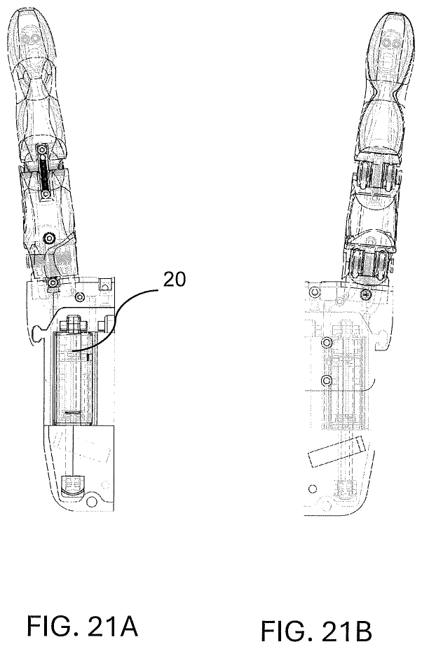

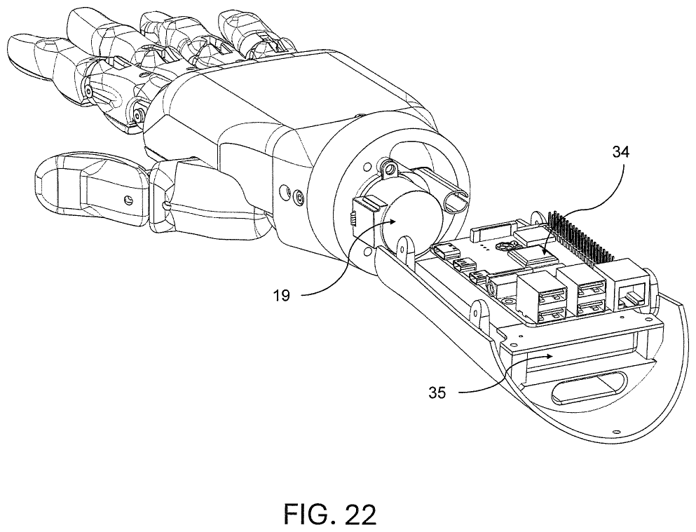

Each finger is driven by its own micro actuator, connected via a durable and lightweight line (30) as shown in FIGS. 11-12 and 17-21B. In some embodiments the line (30) is made of polytetrafluoroethylene (PTFE) or nylon. In each finger, the line extends from the distal phalanx (16), middle phalanx (15), and proximal phalanx (14) along the length of the finger, passes through the finger joint (13) and knuckle joint (10), and terminates in the actuator housing (26). A line divider maintains proper alignment and tension, with adjustable mechanisms in the actuator housing allowing fine-tuning of each finger's movement for optimized performance.

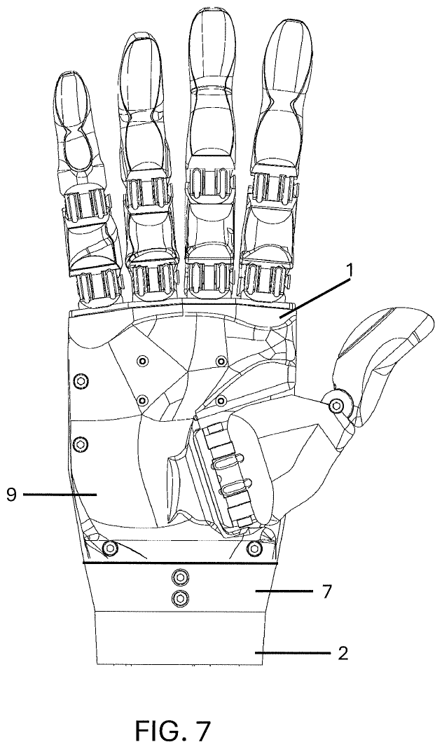

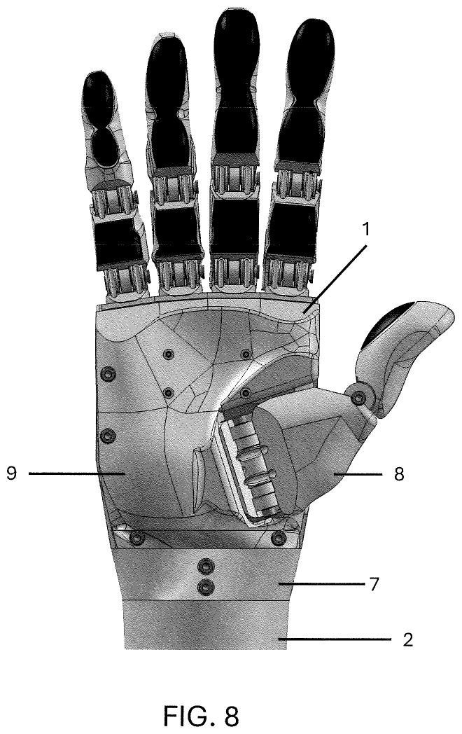

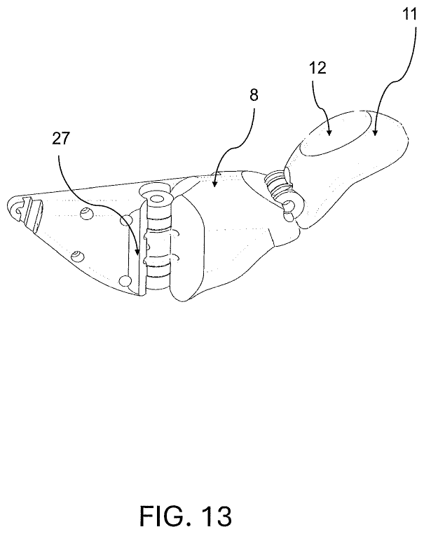

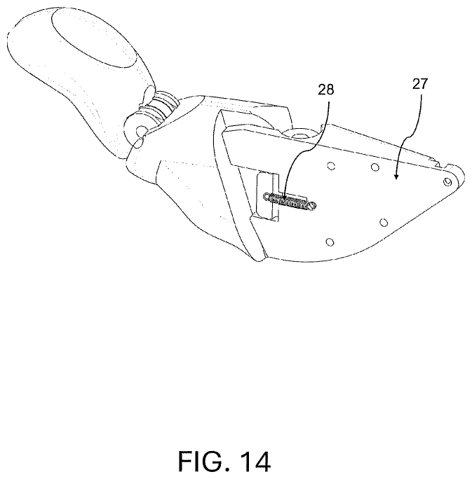

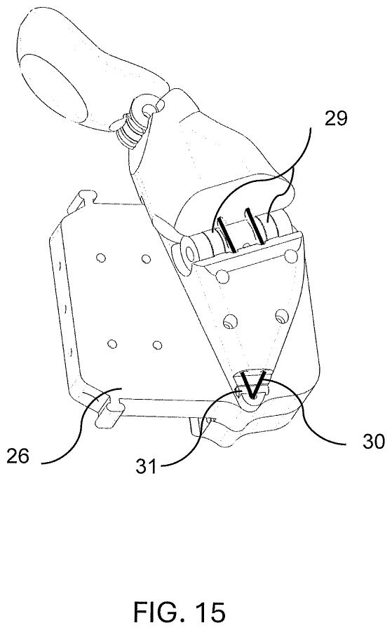

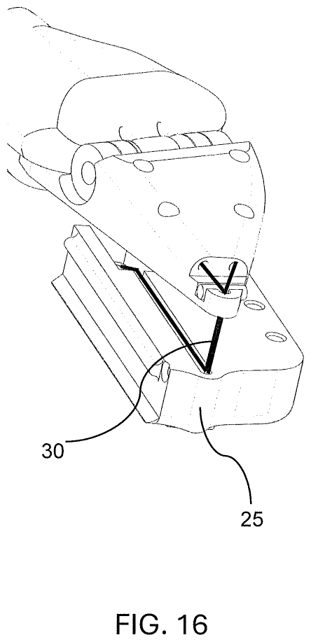

Thumb Design and Actuation (FIGS. 13-16): The thumb is essential for grasping and manipulation, featuring two joints that enhance its range of motion. The first joint connects the distal phalanx (11) of the thumb to the proximal phalanx (8) of the thumb, while the second connects the proximal phalanx (8) to the thenar (27). The thumb's surface incorporates a flexible thermoplastic material forming the fingerprint (12), which is specifically chosen for its softness and flexibility, as shown in FIGS. 7-10 and 19. This material enhances tactile response and allows for accurate sensor integration, enabling the perception of temperature and pressure variations, providing a sensory experience akin to a natural hand.

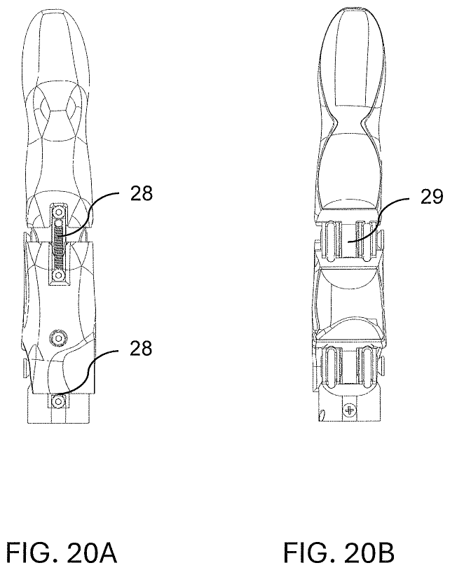

An extension spring (28) automatically retracts the proximal phalanx (8, 14) to its resting position when not in use, ensuring a natural posture, as shown in FIGS. 20A and 20B. The thumb actuation involves a line (30) that follows two distinct paths for smooth operation. The line passes through slots in the knuckles joint (10) and converges through openings in the actuator housings (25, 26) and the thenar region (27), allowing stable and controlled movement.

Actuation and Movement Control (FIGS. 12, 17, 21A and 21B): The hand section (1) features independent micro linear actuators (20-24) for each finger and thumb, providing individualized control for enhanced dexterity. The lines (30) linking the actuators to the fingertips enable smooth, lifelike movements. Manual adjustments to the line tension allow users to customize finger motions for specific tasks, such as gripping, typing, or manipulating objects, ensuring fine control of each digit.

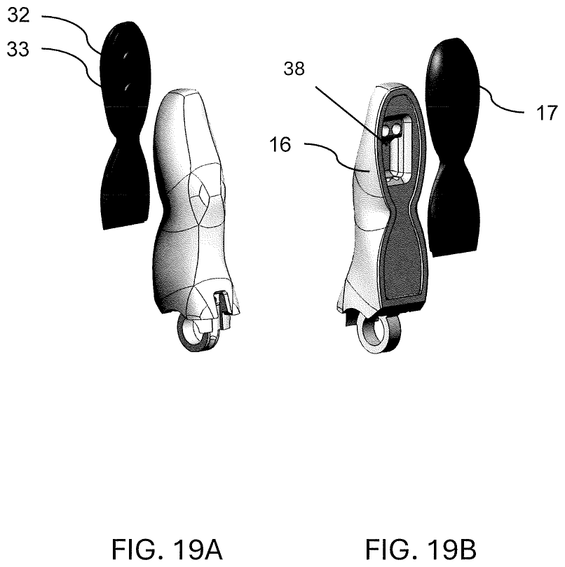

Sensor Integration and Feedback (FIGS. 19A and 19B): The hand section (1) includes a sensory feedback system. The fingerprints (12, 17) on each fingertip are printed using a soft and flexible thermoplastic material, which facilitates the integration of embedded sensors. Embedded sensors (32, 33) are within internal cavities (38) in the fingers, as shown in FIG. 19A/B, with the fingerprint covering the cavity (38) to protect the sensors (32,33) while maintaining flexibility and functionality. These sensors (32, 33) detect tactile forces, temperature, and pressure, delivering real-time haptic feedback to the user. Feedback is delivered to the user via at least one of a flexible heating film and microvibration motors. The heating film simulates the sensation of warmth when the user touches an object determined by the sensors to be above a predetermined temperature (i.e., a hot object), while the vibration motors activate with varying intensities to indicate varying levels of pressure or tactile forces. The system allows the user to perceive and interpret sensory information, enhancing interaction with the prosthetic arm. The flexible material ensures that the sensors maintain close contact with the surface, increasing the sensitivity and accuracy of the sensory feedback. Sensor signals are processed by the microcomputer and transmitted to the user via haptic feedback mechanisms, providing an intuitive and realistic experience.

Materials and Manufacturing: In some embodiments, the hand is produced using 3D printing technology, allowing for complex customization and rapid prototyping. This manufacturing process ensures that components are tailored to the user's specific needs, making the hand lightweight, cost-effective, and modular. The design facilitates easy replacement of parts, such as fingers and wrist joints, enabling quick maintenance and upgrades.

Modular Architecture and Efficiency: The bionic arm's modular design allows components such as hand (1), wrist (2), forearm (3), forearm cover (6), and both elbow (4) and upper arm (5) extensions to be easily attached, removed, or replaced. These extensions provide flexibility to accommodate different types of limb amputations, ensuring the prosthetic is versatile and adaptable to various user needs. The forearm cover (6) is designed to provide convenient access to the battery (35) and microcomputer (34), allowing for easy programming or maintenance. Lightweight actuators and optimized mechanical linkages ensure efficient power consumption while providing natural movement and realistic joint motion. This modular architecture supports routine maintenance and future upgrades, minimizing downtime and improving the prosthetic's overall longevity.

In a second embodiment, the prosthetic limb incorporates a multi-material fused structure printed in a single manufacturing cycle, hereinafter referred to as the FusionLimb configuration. This structure integrates rigid and flexible regions without the need for discrete fasteners, bearings, or springs.

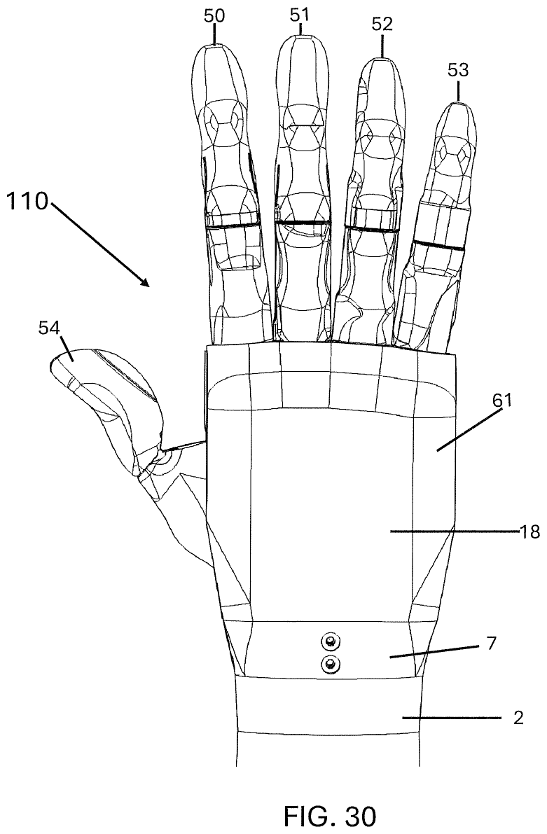

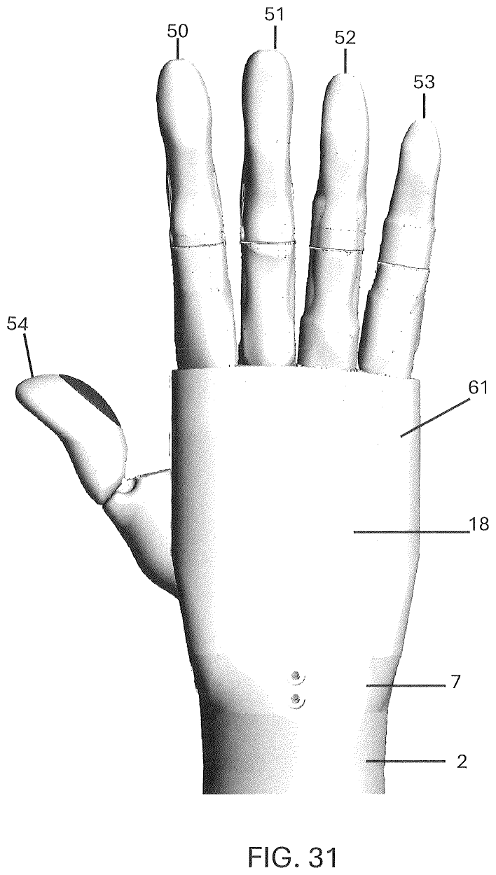

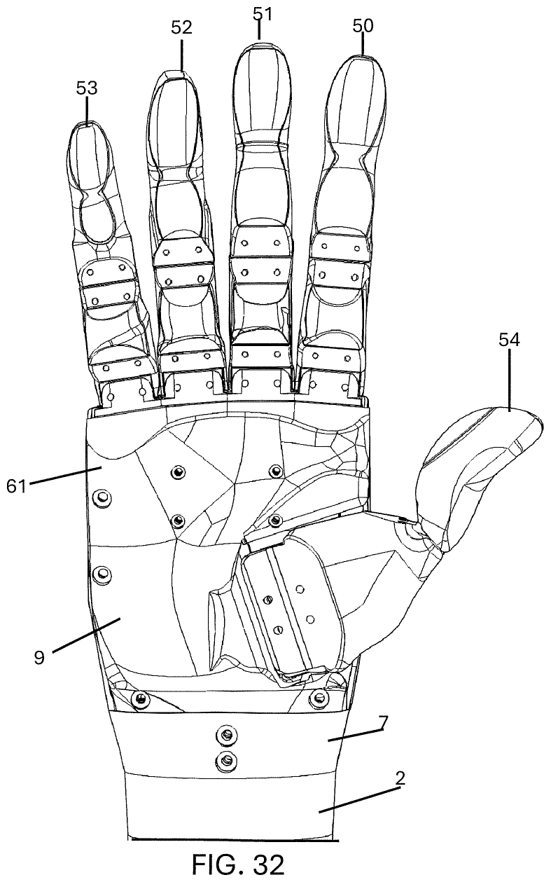

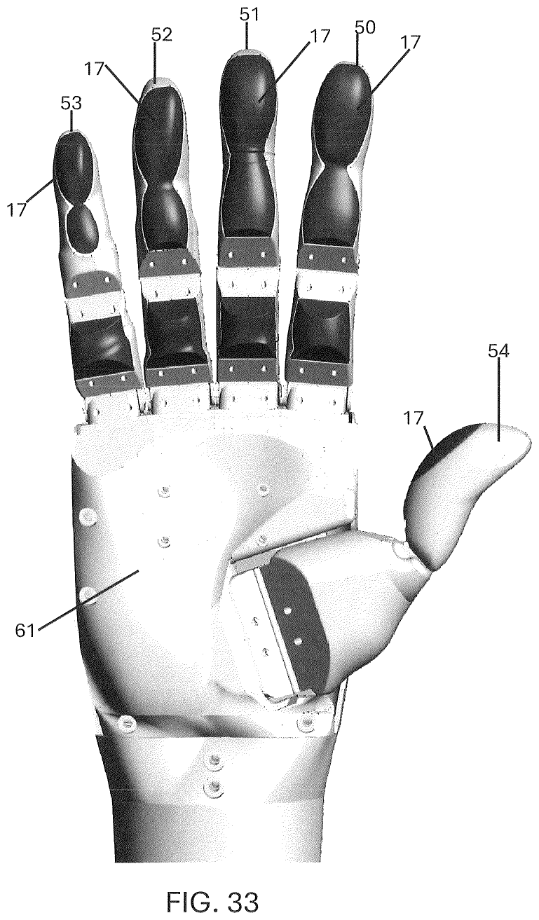

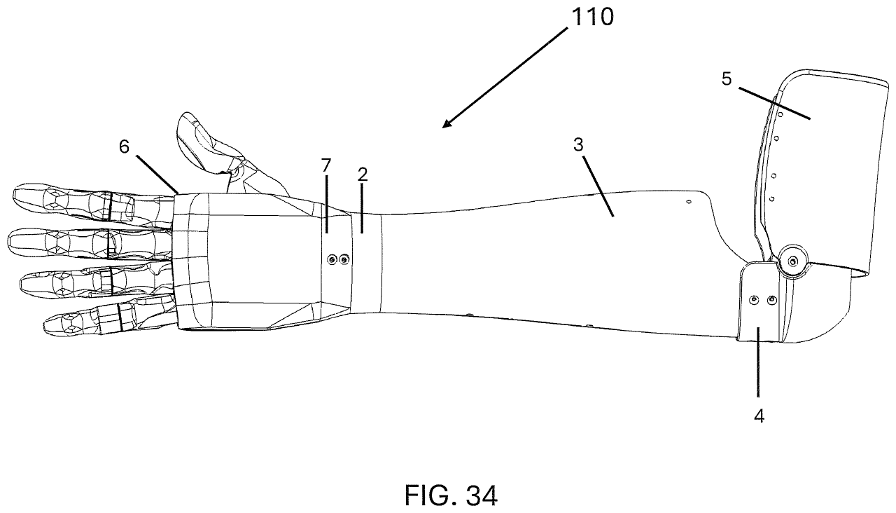

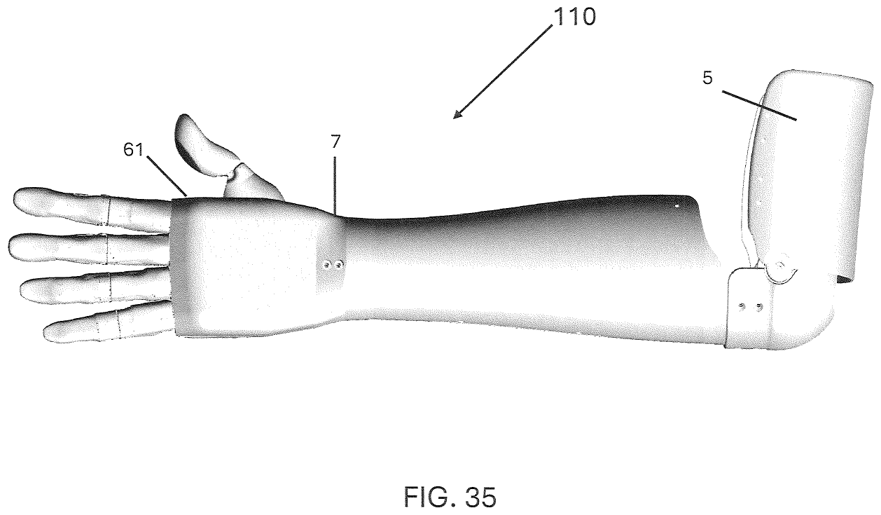

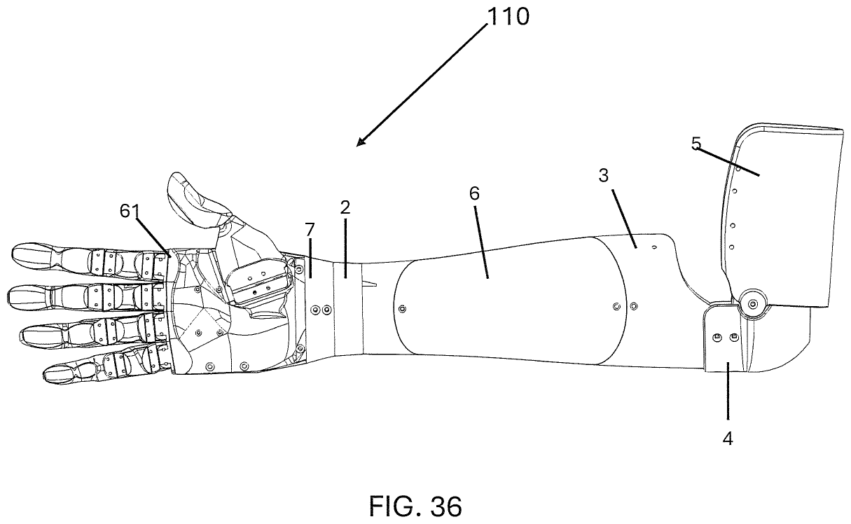

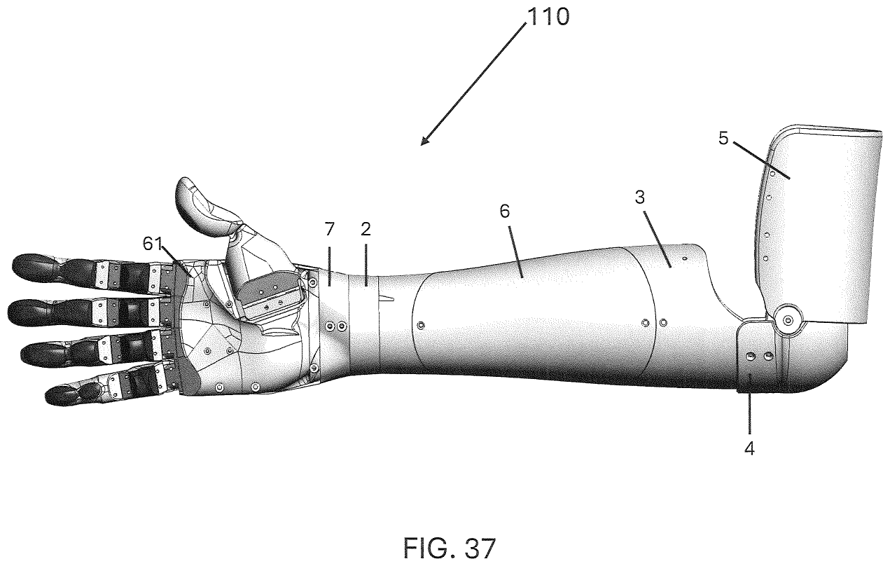

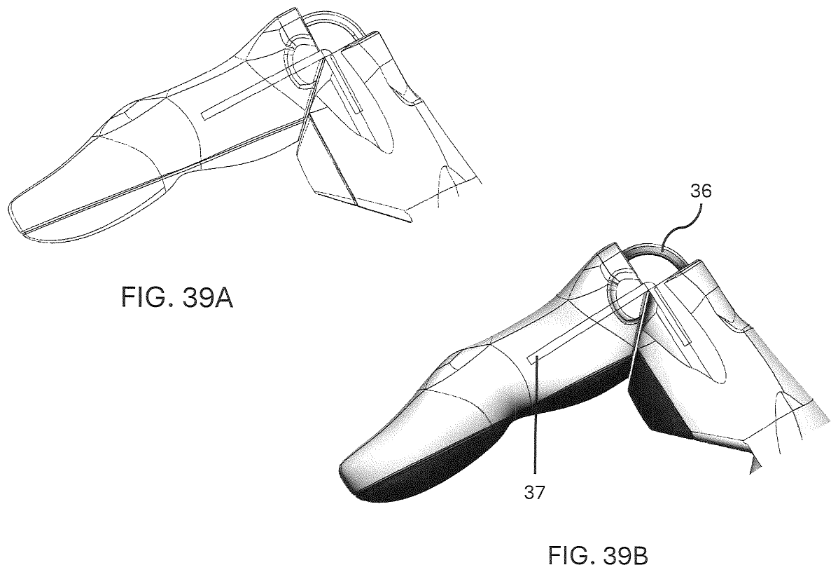

In this second embodiment, shown in FIGS. 30-39B, the four fingers, excluding the thumb, are produced as a single continuous printed component comprising a rigid load-bearing material (e.g., carbon-fiber reinforced nylon or BioMed) co-printed with a flexible thermoplastic polyurethane (TPU 95A HF). This integration eliminates discrete phalangeal segments and associated mechanical connectors such as screws, nuts, and springs. The resulting finger assembly provides compliant joint motion, increased structural rigidity, and reduced assembly time.

The FusionLimb embodiment provides several performance advantages over prior designs: Weight Reduction: Removing traditional joint hardware results in a lighter prosthesis, reducing user fatigue. Enhanced Reliability: Fewer mechanical parts means fewer potential points of failure. Smooth, Natural Motion: Material pairing allows for controlled flexion and extension without mechanical noise. Faster Production: Single-pass printing cuts manufacturing and assembly time. Simplified Maintenance: One-piece elements can be replaced more quickly and easily than multi-part assemblies.

In the FusionLimb embodiment, the thumb is likewise produced as a monolithic structure, co-printed in one continuous build process from rigid and flexible materials. This design replaces prior multi-part assemblies with a single part, improving durability and simplifying replacement. TPU 95A HF as the flexible material in joint regions, and carbon fiber-reinforced nylon 6 (PA6-CF) or BioMed-grade polymer for rigid segments, co-printed during a single build. The thumb is transitioned from a three-part assembly to a one-piece structure with integrated flexibility zones.

In the FusionLimb embodiment, the four fingers (50-53) (excluding the thumb) are printed as a single continuous component, and the thumb (54) is likewise monolithic, with the performance advantages previously described. All other structural components, including the palm (9), wrist (2), forearm (3) and forearm cover (6), elbow (4), and upper arm (5), remain the same as in the first embodiment. The FusionLimb also retains the same internal architecture as described in the first embodiment, including the micro linear actuators (20-24), microcomputer (34), battery (35), connecting lines, and sensors (32-33).

The principal distinction between the first (100) and second (110) embodiments is that FusionLimb uses a single-pass, integrated multi-material printing process, which eliminates the need for separate springs, screws, and nuts. Instead, small zones of flexible yet durable material are fused into the finger structure, enabling natural flexion without mechanical springs. This provides smoother, more accurate motion with reduced mechanical complexity.

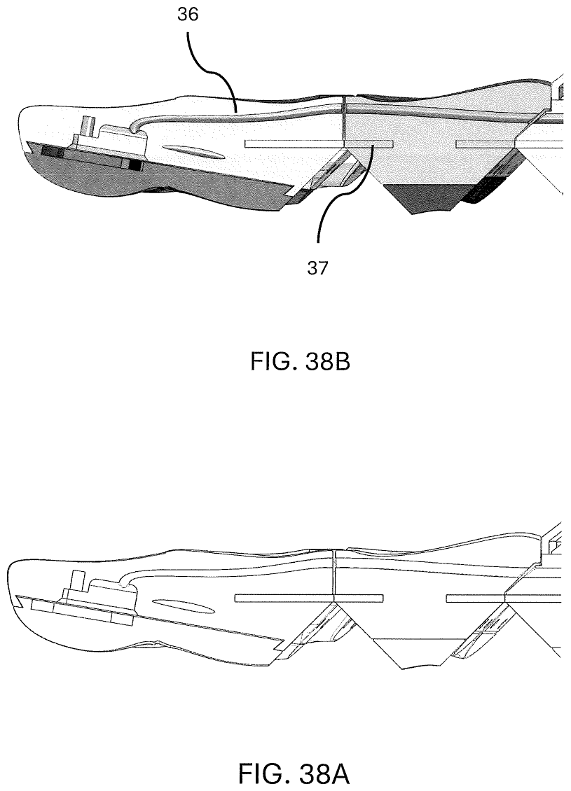

FIG. 38A and B and 39A and B show the flexible material parts printed using TPU 85A (No 36: Restorative Band) and TPU 95A (No 37: Joint Flex Connector) integrated into a finger. Joint Flex Connector 37 acts as a flexible hinge material between two phalanges. It provides the mechanical connection that allows bending at the joint while maintaining structural integrity. Restorative Band 36 provides elastic restoring force to the finger. After the actuator bends the finger, the band pulls it back to the initial straight position once actuation is released. This replaces traditional metal return springs. The other internal component drawings remain unchanged from the first embodiment (100).

The material advantages for the FusionLimb embodiment are that TPU 95A HF provides compliant, skin-like motion surfaces and that PA6-CF or resin-based biocompatible material offers high stiffness, durability, and biocompatibility.

The performance improvements with the FusionLimb embodiment are in weight reduction, enhanced reliability, smooth and natural motion, faster production, and simplified maintenance. Weight reduction is achieved by removing traditional joint hardware results in a lighter prosthesis, reducing user fatigue. Enhanced reliability is achieved by having fewer mechanical parts which means fewer potential points of failure. Smooth and natural motion is achieved by material pairing which allows for controlled flexion and extension without mechanical noise. Faster production is achieved by single-pass printing which cuts manufacturing and assembly time. Simplified maintenance is achieved as one-piece elements can be replaced more quickly and easily than multi-part assemblies.

Control System and Multi-Modal Input Integration: The prosthetic arm features a microcomputer (34) as the central control unit, which processes input from various sources, including EEG-based brain-computer interfaces and, optionally, EMG-based systems and manual switches. The control system coordinates the activation of micro linear motors, adjusts power distribution, and integrates real-time data from the sensory feedback system to optimize performance. Users can switch between or combine multiple input methods for flexible and adaptable control.

Data Augmentation and Transfer Learning: The use of GANs for synthetic data generation helps address the challenges of data scarcity and EEG signal variability. GANs create synthetic EEG samples to expand the training dataset, enhancing the hybrid neural network's ability to generalize across different users. Transfer learning fine-tunes the model for individual users, improving classification accuracy and ensuring robust performance in diverse environments.

Affordability and Non-Invasiveness: Unlike traditional EMG-based systems, which can be costly and invasive, this invention provides a non-invasive, brain-controlled solution that is more accessible and cost-effective. By using EEG signals instead of muscle-based control, the system eliminates issues related to muscle fatigue and signal reliability, offering a more robust interface.

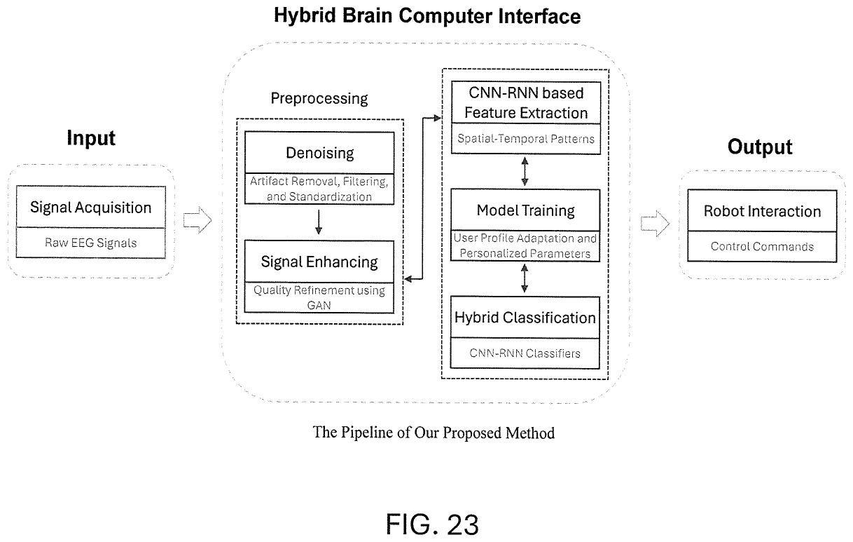

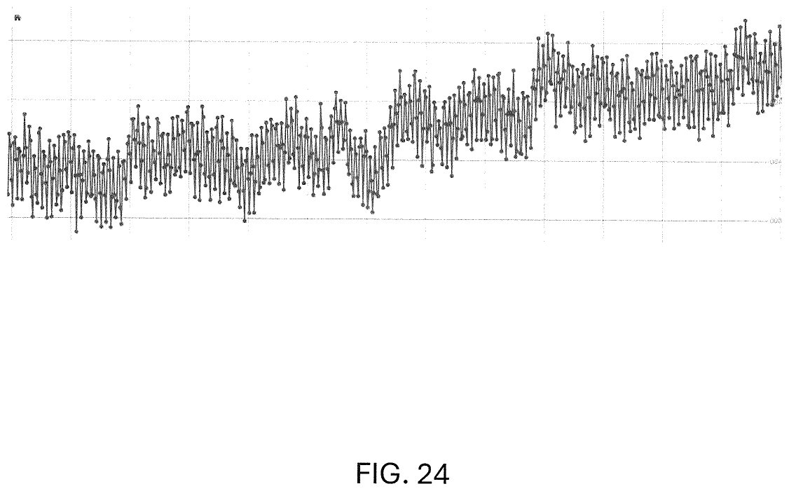

The Hybrid Brain-Computer Interface (HBCI) design shown in FIG. 23 incorporates a CNN+RNN architecture that uses transfer learning for EEG signal processing. A representative EEG signal is depicted in FIG. 24. The architecture consists of three main components: (1) Pre-trained CNN for feature extraction, (2) RNN for modeling temporal dependencies, and (3) Transfer Learning and Fine-tuning Mechanism.

The pre-trained CNN is the first component. It processes structured grid data, such as images, by learning spatial hierarchies of features. CNNs are widely applied in fields such as image recognition and EEG signal processing due to their ability to exploit the spatial structure of data.

The primary components of a CNN include:
(1) Convolutional Layers: These layers are the foundational elements of CNNs. A convolutional layer applies multiple filters (kernels) to the input data, performing a mathematical convolution operation. Each filter slides over the input and extracts specific features (e.g., edges, textures) at different levels of abstraction.

(2) Pooling Layers: Pooling layers downsample the feature maps to reduce their dimensionality, thereby decreasing computational complexity and making the model more robust to small spatial variations in the input.

(3) Fully Connected (Dense) Layers: Following the convolutional and pooling layers, the high-level abstracted features are passed to fully connected layers. These layers connect every neuron in one layer to every neuron in the next, similar to traditional neural networks.

(4) Flattening: The output from the last convolutional or pooling layer is a multi-dimensional feature map. Flattening converts this feature map into a one-dimensional vector, which is then fed into the fully connected layers for classification or other tasks.

In this invention, three layers of 1D CNN are employed. The use of 1D CNNs reduces the computational resources required to train the model, making it suitable for deployment on portable devices such as a Raspberry Pi or NVIDIA Jetson Nano. Additionally, the training process is significantly faster with 1D CNNs.

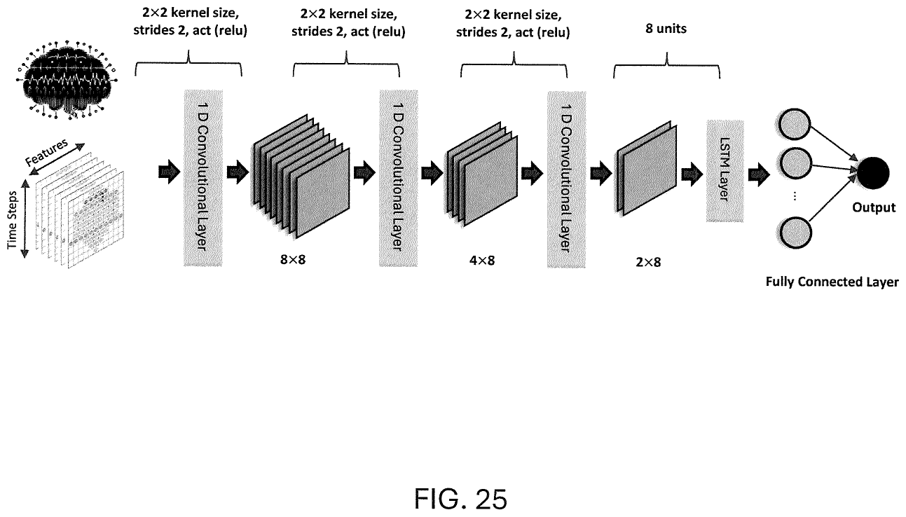

FIG. 25 illustrates the detailed architecture of the CNN+RNN model used in this invention. The CNN component comprises four layers:
(1) Input Layer: i=Input(shape=(16, 1)). This layer expects a sequence of length 16, where each time step contains a single feature (e.g., time-series data or EEG channels).
(2) First Convolutional Layer: x=Convolution1D(8, kernel_size=2, strides=2, activation=‘relu’)(i). This 1D convolutional layer uses 8 filters, a kernel size of 2, and a stride of 2. The kernel size of 2 allows the layer to examine 2 adjacent time steps simultaneously, while the stride of 2 means the kernel moves over the input by skipping one time step at each step, effectively halving the sequence length. As a result, the original sequence length of 16 is reduced to 8, with each filter capturing different local patterns within the input. The use of the ReLU (Rectified Linear Unit) activation function introduces non-linearity, allowing the model to learn more complex representations.
(3) Second Convolutional Layer: x=Convolution1D(8, kernel_size=2, strides=2, activation=‘relu’)(x). This layer, configured similarly to the first, employs 8 filters, a kernel size of 2, and a stride of 2. As the kernel slides over the input, the sequence length is further reduced from 8 to 4, continuing to downsample the data. This layer allows the model to learn additional local patterns from the downsampled input, using the 8 filters to extract higher-level features from the sequence.
(4) Third Convolutional Layer: x=Convolution1D(8, kernel_size=2, strides=2, activation=‘relu’)(x). This layer, with 8 filters, a kernel size of 2, and a stride of 2, further reduces the sequence length from 4 to 2. By operating over the downsampled input, it continues to extract higher-level features, capturing more abstract patterns from the data. The use of 8 filters allows the layer to analyze the input over larger time windows, enhancing the model's ability to identify complex temporal relationships.

The subsequent component is the RNN for temporal dependency modeling. RNNs are designed to handle sequential data where the order of the data points is significant. Unlike feedforward neural networks, RNNs maintain a “memory” of previous inputs through hidden states, making them well-suited for tasks such as time series analysis, language modeling, and speech recognition. This invention utilizes Long Short-Term Memory (LSTM) units, a type of RNN specifically designed to overcome the vanishing gradient problem, enabling the network to learn long-term dependencies. LSTMs incorporate gates (input, forget, and output) that regulate the flow of information, determining what should be remembered or forgotten.

FIG. 25 also details the LSTM configuration:
LSTM Layer: x=LSTM(8, return_sequences=False, return_state=False)(x). This LSTM layer contains 8 units, producing an 8-dimensional output vector that summarizes the learned temporal patterns. The input to the LSTM is the output of the last convolutional layer, now a sequence of length 2 with 8 features per time step. return_sequences=False indicates that the LSTM outputs a single 8-dimensional vector representing the entire sequence, while return_state=False ensures only the LSTM output is used.

Finally, a Dense (Fully Connected) Layer connects the LSTM's output to a single output neuron: y=Dense(1)(x). The final output is a single scalar value.

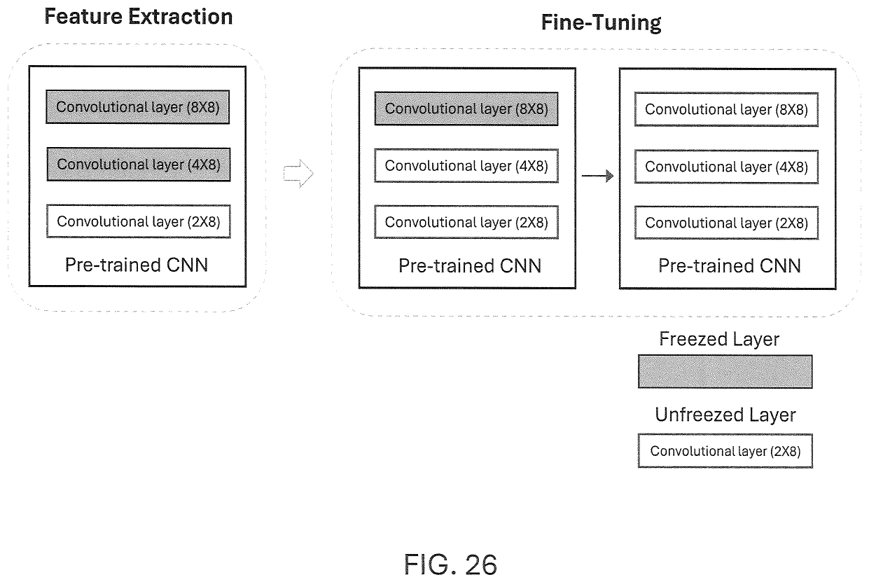

In this invention, transfer learning is employed, as shown in FIG. 26, to fine-tune the pre-trained CNN. Transfer learning allows a model trained on one task (source) to be reused or fine-tuned for a different, related task (target). This approach leverages knowledge from the source task to improve generalization on the target task, especially when the target data is limited. Transfer learning is particularly effective in scenarios where gathering large amounts of training data is challenging or expensive.

The transfer learning mechanism, shown in FIG. 26, is employed to fine-tune the pre-trained CNN, adapting it to the specific characteristics of the EEG dataset. This process involves two main strategies: feature extraction and fine-tuning. In the feature extraction approach, the CNN is used primarily as a feature extractor with minimal or no fine-tuning, where most layers remain frozen, and only the final layers are updated to adapt to the new data. In the fine-tuning approach, layers of the pre-trained CNN are gradually unfrozen and fine-tuned based on validation performance, allowing the model to learn more relevant features from the EEG data while maintaining stability. At the same time, the RNN layers are trained from scratch to capture temporal dependencies within the data. A low learning rate is used during fine-tuning to ensure that weight updates do not disrupt the pre-trained model's learned representations.

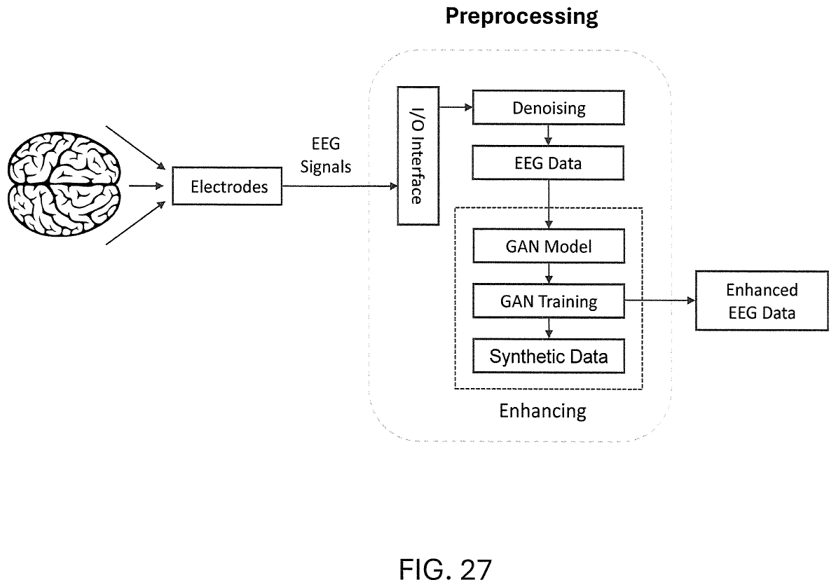

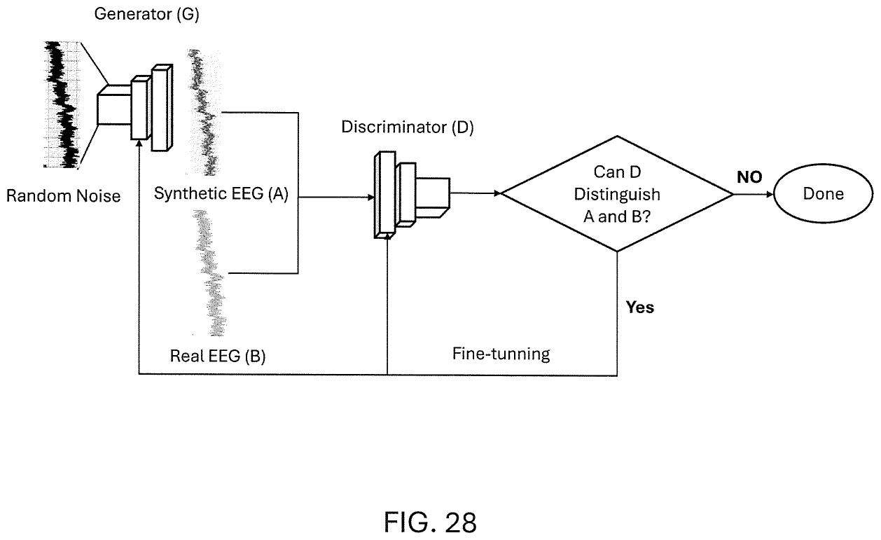

In this invention, an adversarial network, as shown in FIG. 27, is used for upsampling the EEG dataset through the implementation of a Generative Adversarial Network (GAN). As detailed in FIG. 28, the GAN consists of two neural networks: a generator and a discriminator, which operate in opposition during training. The generator creates synthetic EEG signals from random noise, progressively improving its ability to produce realistic data that mimics the distribution of real EEG samples. Meanwhile, the discriminator is trained to differentiate between authentic EEG signals from the original dataset and the synthetic signals produced by the generator, learning to identify the features that make EEG data appear genuine.

The generator and discriminator engage in adversarial training, where the generator attempts to fool the discriminator into classifying synthetic data as real, while the discriminator continuously improves its ability to distinguish real from synthetic signals. This iterative training process enhances the quality of the generated samples. Once the GAN is trained, the generator produces a larger set of synthetic EEG signals, which can be used to augment the original dataset, balance class distributions, or expand the dataset size. The augmented dataset, now containing both real and synthetic samples, can then be used to train the CNN+RNN model or other machine learning models for improved performance.

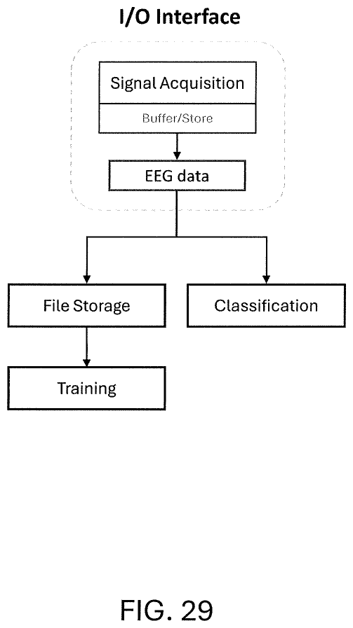

The I/O interface, illustrated in FIG. 29, oversees the signal acquisition process by buffering and storing EEG signals obtained from a neurodetection device, namely, a skullcap including a plurality of EEG signal-detecting electrodes worn on the user's head. The skullcap communicates with the prosthetic via a Bluetooth connection. EEG signals are collected from electrodes on the skullcap and transmitted through a connection board. On the prosthetic side, a receiver connected to the microcomputer receives these signals. The microcomputer's I/O interface processes the incoming signals, which can be buffered or stored in different mediums, such as computer memory or files. In this invention, the signals are stored in CSV files, with separate files organized according to the EEG data corresponding to different arm movements. The I/O interface streamlines the dataset collection process for each individual. The CNN+RNN model is trained on datasets gathered from multiple individuals. Once trained, the model is used for classification tasks. During operation, the EEG dataset is loaded into memory and fed into the model. The model processes these input signals to predict corresponding outputs, leveraging its architecture to achieve precise and reliable classification.

The EEG signals are collected from multiple regions of the brain, including the frontal lobe, parietal lobe, occipital lobe, and temporal lobe, using electrodes embedded within the skullcap. These electrodes are strategically placed to capture neural activity associated with different brain functions. Each hemisphere of the brain is equipped with eight signal channels, resulting in a total of 16 channels across both hemispheres. The signals collected by the electrodes are numerical values representing the strength of electrical activity (voltage fluctuations) at specific locations on the scalp. This data is continuously recorded in microvolts (μV), reflecting the brain's electrical dynamics in real-time. The 16 signals serve as features for subsequent analysis. These features capture spatial information about brain activity across different regions, allowing for effective classification of cognitive or motor states during machine learning tasks. This setup ensures comprehensive coverage of the brain's electrical activity and provides robust input data for processing and classification CNN+RNN models.

The input signals are classified as Imaginary EEG signals, which are brainwave data generated when an individual imagines performing a specific action or movement without physically executing it. These signals originate from motor imagery, a cognitive process where an individual visualizes or mentally rehearses a movement. During motor imagery, the motor cortex and premotor cortex become active, simulating the neural activity associated with actual movement. This activity produces electrical signals that can be captured and measured using EEG devices, such as electrodes embedded in a skullcap. These electrodes detect and record the subtle voltage fluctuations on the scalp, representing the brain's electrical activity during the imagined movement.

Imaginary EEG signals are generated by the brain's motor cortex and supplementary motor areas, mimicking the neural activity of actual movements without involving muscles or physical motion. These signals exhibit distinct patterns, including Mu Rhythm (8-13 Hz), which decreases during motor imagery, Beta Rhythm (13-30 Hz), associated with motor planning, and Event-Related Desynchronization (ERD) and Synchronization (ERS), reflecting activation and relaxation of motor areas. Signal strength depends on the individual's motor imagery ability, mental focus, and level of training. Using non-invasive recording techniques, these signals are captured via electrodes placed on the scalp at positions aligned with the motor cortex, enabling accurate detection for applications such as brain-computer interfaces and neurorehabilitation.

The following disclosure sets forth an example of how EEG signals can be acquired, preprocessed, and prepared for machine-learning classification. The system is not limited to any specific commercial EEG headset; rather, different types of non-invasive EEG headsets or electrode arrangements may be used as long as they are configured to capture scalp potentials and provide the signals for processing. Also, while other specific equipment is listed being used in processing signals to be input into the CNN+RNN model, this disclosure is not limited to that specific equipment as other similar equipment can be used.

In one example, a non-invasive EEG headset is used to position electrodes on the scalp following the 10-20 system (e.g., Fp1, Fp2, C3, Cz), with each electrode configured to measure microvolt-level voltage differences relative to a reference electrode. The headset also includes a reference electrode (REF), typically placed at a neutral location like the mastoid or earlobe, and a bias/ground electrode (BIAS) that injects a small signal to reduce noise and improve common-mode rejection. These electrodes connect to a multichannel EEG acquisition module that includes an analog front-end circuit configured to amplify and digitize scalp potentials. Each channel operates in a differential configuration, measuring the potential difference between its electrode and a reference signal. The analog front-end provides low-noise amplification (e.g., on the order of 20-30×) to accommodate the extremely small amplitude of EEG signals (typically 1-100 μV) and digitizes them with high resolution, such as a 24-bit analog-to-digital converter. The sampling rate may be configured, for example, at 250 Hz or 125 Hz, with the digitized samples transmitted to a computer in real time via wired or wireless communication. This acquisition chain—electrode→reference/ground→amplification→high-resolution digitization→wired/wireless data transfer—preserves the raw EEG voltages for subsequent signal processing.

Each channel's output from the headset is a signed voltage value in microvolts, representing the instantaneous potential difference between the electrode and the reference. Positive values mean the electrode's voltage is higher than the REF; negative values mean it is lower. For example, a Ch1 value of −12.3 μV at Fp1 indicates the electrode is 12.3 μV lower than the reference, while a Ch2 value of 8.7 μV at Fp 2 means it is 8.7 μV higher. These raw values reflect the summed postsynaptic potential of millions of neurons beneath each electrode, but they do not directly indicate specific thoughts or actions. Instead, they are characterized by brainwave frequency bands—Delta (0.5-4 Hz, deep sleep), Theta (4-8 Hz, drowsy/meditative), Alpha (8-13 Hz, relaxed), Beta (13-30 Hz, active thinking), and Gamma (>30 Hz, high cognitive processing). Because EEG data is inherently noisy, preprocessing steps such as filtering, artifact removal (eye blinks, muscle activity, 60 Hz mains interference), and normalization are conducted before extracting features like band power. These features can then be fed into our CNN+RNN model to detect patterns or classify mental states.

The values captured from the 8 channels represent dynamic electrical activity generated by the brain's cortical neurons, captured at specific scalp locations and expressed as tiny voltage fluctuations over time. These fluctuations arise primarily from the summed postsynaptic potentials of large populations of neurons firing synchronously, rather than from single neurons. While the raw numbers themselves cannot directly tell you “What you are thinking” or “what movement you are planning,” they form the foundational data from which meaningful patterns can be derived. We need to transform those values into interpretable features—such as power in specific frequency bands or event-related potentials—that can reveal aspects of cognitive state, sensory processing, or motor intention.

In order to transform those values into interpretable features, we used the bandpass filter to process the signal values. The strategy is to define a frequency range that preserves the desired brainwave components while attenuating frequencies outside that range. In the implementation, we used a 4th-order Butterworth bandpass filter with a passband of 0.5-50 Hz. We use Python's SciPy library with the butter function to design the filter and filtfilt to apply it. First, we define the lower and upper cutoff frequencies—0.5 Hz and 50 Hz—along with the sampling rate (250 Hz) and the filter order (4th-order Butterworth). The cutoff frequencies are normalized by dividing each by half the sampling rate (the Nyquist frequency) before passing them to butter, which returns the filter coefficients. These coefficients are then used by filtfilt to process the raw channel voltage values (in microvolts), producing a filtered signal that retains only components between 0.5 Hz and 50 Hz. This process removes slow drifts and high-frequency noise while preserving the EEG's core brainwave information.

Then in order to remove the impact from the artifact impacts, such as, eye-blink or muscle activity and interference from the lights and power lines. We applied a notch filter, which targets and attenuates a very narrow frequency band while leaving the rest of the signal largely unaffected. We designed a notch filter using SciPy's iirnotch function with a target frequency and a quality factor, which determines how narrow the notch will be. The iirnotch function returns the filter coefficients, which are then applied to the raw EEG channel voltages using filtfilt for zero-phase distortion. Finally, we have more reliable and cleaner neural signals.

The last step before we input the signals into our CNN+RNN model is using the transformer model to enhance the signal values. This process involves leveraging transformer model's sequence modeling capabilities to clean, reconstruct, and extract more informative features from EEG data, which is essentially a multichannel time-series. The reason why to use transformer is that transformers can be adapted to model both long-range temporal dependencies—capturing relationships across distant time points in patterns like motor imagery or event-related potentials—and cross-channel interactions, where self-attention learns how signals from different electrodes relate to each other, enhancing correlated brain activity while suppressing noise. Transformers can also perform data-driven denoising to reconstruct clean signals from noisy input. The workflow in our work is below. It includes preparing the data by filtering (in previous step). Then formatting the input so each channel's temporal sequence is treated as a token, with positional encoding to preserve time order. In the implementation, the transformer model is built as a Transformer Encoder designed to process multichannel EEG signals with 8 channels and a sequence length time point. First, the raw EEG input of shape (batch size, sequence length, channels) is projected into a higher-dimensional space (64 features) using a linear layer, allowing the transformer to better capture complex patterns. A learnable positional encoding is added to the input to provide the model with information about the temporal order of the data, since transformers themselves don't inherently understand sequence position. The data is then permuted to shape (sequence length, batch size, features) to fit the PyTorch transformer input format and passed through two layers of multi-head self-attention within the Transformer Encoder, which learns temporal dependencies and inter-channel relationships. After encoding, the output is projected back from the transformer's feature space to the original 8-channel EEG space using another linear layer, producing an enhanced version of the input signals. During training, the model receives noisy simulated EEG data (clean EEG plus added noise) and optimizes its parameters to minimize the mean squared error between its output and the clean EEG signals, effectively learning to denoise and enhance the brain signals. The final output is prepared as a 2D array for the next step as the input for our CNN+RNN model.

The input to the CNN+RNN model is structured as a series of 2D arrays, each representing preprocessed time-series EEG signals recorded along with their corresponding timestamps. The model utilizes a signal collection window (duration) of 5 seconds, a duration chosen for its ability to provide optimal performance while matching the time required for the prosthetic to complete a single movement. During this interval, EEG signals are continuously collected, forming a cohesive data unit that encapsulates the complete sequence of neural activity associated with an entire movement. Then the signals are preprocessed as above by the filters and transformer model. By structuring the input in this manner, the model can effectively learn and analyze the temporal and spatial dependencies within the signals, enabling accurate recognition and classification of the imagined movements. This approach ensures that each input unit is comprehensive, capturing the full dynamics of the motor imagery process.

The disclosed control system may be embodied in computer program instructions stored on a non-transitory computer readable storage medium configured to be executed by a computing system, such as, for example, a microcomputer embedded in the prosthetic arm. The computing system utilized in conjunction with the computer-aided system described herein will typically include a processor in communication with a memory, and a network interface. Power, ground, clock, and other signals and circuitry are not discussed, but will be generally understood and easily implemented by those ordinarily skilled in the art. The processor, in some embodiments, is at least one microcontroller or general purpose microprocessor that reads its program from memory. The memory, in some embodiments, includes one or more types such as solid-state memory, magnetic memory, optical memory, or other computer-readable, non-transient storage media. In certain embodiments, the memory includes instructions that, when executed by the processor, cause the computing system to perform a certain action. Computing system also preferably includes a network interface connecting the computing system to a data network for electronic communication of data between the computing system and other devices attached to the network. In certain embodiments, the processor includes one or more processors and the memory includes one or more memories. In some embodiments, computing system is defined by one or more physical computing devices as described above. In other embodiments, the computing system may be defined by a virtual system hosted on one or more physical computing devices as described above.

The foregoing detailed description is given primarily for clearness of understanding and no unnecessary limitations are to be understood therefrom for modifications can be made by those skilled in the art upon reading this disclosure and may be made without departing from the spirit of the invention.

1. A prosthetic arm system comprising a prosthetic arm apparatus, the prosthetic arm apparatus comprising:
a plurality of interconnected sections arranged adjacent to each other, the sections including a hand section connected to a wrist connector, the wrist connector connected to a wrist section, the wrist section connected to a forearm section;
wherein the hand section includes
a palm section connected to the wrist connector;
a knuckles joint connected to the palm section;
a plurality of fingers connected to the knuckles joint;
a thumb connected to the palm section; and
a plurality of actuators;
wherein each of the plurality of fingers includes
a proximal phalanx connected to the knuckles joint,
a middle phalanx connected to the proximal phalanx,
a distal phalanx connected to the middle phalanx, where each of the proximal, middle, and distal phalanges is covered by a flexible thermoplastic material, and wherein the distal phalanx includes an internal cavity having at least one sensor positioned within the internal cavity; and
a line extending from the distal phalanx, through the middle phalanx, through the proximal phalanx, and operatively connected to one of the plurality of actuators in the hand section;
wherein the thumb includes
a thenar connected to the palm section,
a thumb proximal phalanx connected to the thenar, and
a thumb distal phalanx connected to the thumb proximal phalanx;
a line extending from the thumb distal phalanx, through the thumb proximal phalanx, through the thenar, and operatively connected to one of the plurality of actuators in the hand section;
wherein the forearm section includes a microcomputer and a battery, wherein the plurality of actuators are in wired or wireless communication with the microcomputer and the battery.

2. The prosthetic arm system of claim 1, wherein activation of the actuators by the microcomputer retracts or extends the lines, transitioning the hand between an open state and a closed state.

3. The prosthetic arm system of claim 1, wherein the forearm section is connected to an elbow section, which is further connected to an upper arm extension.
The prosthetic arm system of claim 1, further including a switch which permits for manual opening and closing of the hand.

5. The prosthetic arm system of claim 1 further including a control system for the prosthetic arm, the control system comprising:
at least one non-transitory computer readable storage medium having computer program instructions stored thereon; and
at least one processor configured to execute the computer program instructions causing the processor to perform the following operations:
receive electroencephalography (EEG) signals from a user of a neurodetection device;
segment the received EEG signals by a collection duration to produce segmented EEG signals;
preprocess the segmented EEG signals by bandpass filtering, notch filtering, and transformer-based denoising to produce preprocessed EEG signals;
decode, using a machine-learning system, the preprocessed EEG signals to detect imagined spatial motion of the user, where the imagined spatial motion corresponds to desired motion commands of the prosthetic arm;
execute, using one or more of the plurality of actuators, detected imagined spatial motion of the user;
wherein the machine-learning system is a hybrid system including a first neural network and a second neural network, wherein the received EEG signals are input to the first neural network, wherein output of the first neural network is input to the second neural network, and wherein output of the second neural network is classification and identification of imagined spatial motion.

6. The prosthetic arm system of claim 5, wherein the neurodetection device includes a plurality of EEG signal-detecting electrodes worn on the user's head.

7. The prosthetic arm system of claim 5, further including a switch which permits for manual opening and closing of the hand.

8. The prosthetic arm system of claim 1 where the prosthetic arm is brain-controlled by use of:
a neurodetection device configured to acquire electroencephalography (EEG) signals from a user;
a preprocessing module configured to filter and denoise the EEG signals;
a hybrid neural network decoder configured to classify neural intent from the EEG signals;
a control unit housed in the prosthetic arm and configured to activate one or more actuators of the prosthetic arm in response to the classified neural intent.

9. A prosthetic arm system comprising a prosthetic arm apparatus comprising:
a plurality of interconnected sections arranged adjacent to each other, the sections including a hand section connected to a wrist connector, the wrist connector connected to a wrist section, the wrist section connected to a forearm section;
wherein the hand section includes:
a palm section connected to the wrist connector;
a plurality of fingers connected to the palm section;
a thumb connected to the palm section; and
a plurality of actuators, each finger and the thumb having at least one actuator associated therewith;
wherein the plurality of fingers are produced as a single continuous printed component comprising a rigid load-bearing material, each finger having a joint flex connector which acts as a flexible hinge material and provides a mechanical connection that allows bending at a finger joint while maintaining structural integrity, each finger further having a restorative band which provides elastic restoring force to the finger, where after the associated actuator bends the finger, the restorative band pulls it back to the initial straight position once actuation is released;
wherein the thumb is produced as a single continuous printed component comprising a rigid load-bearing material, the thumb having a joint flex connector which acts as a flexible hinge material and provides a mechanical connection that allows bending at a thumb joint while maintaining structural integrity, the thumb further having a restorative band which provides elastic restoring force to the thumb, where after the associated actuator bends the thumb, the restorative band pulls it back to the initial straight position once actuation is released;
wherein the forearm section includes a microcomputer and a battery, wherein the plurality of actuators are in wired or wireless communication with the microcomputer and the battery.

10. The prosthetic arm system of claim 9 wherein activation of the actuators by the microcomputer retracts or extends the lines, transitioning the hand between an open state and a closed state.

11. The prosthetic arm system of claim 9 wherein each of the plurality of fingers includes
a proximal phalanx connected to a knuckles joint, the knuckles joint connected to the palm;
a middle phalanx connected to the proximal phalanx;
and a distal phalanx connected to the middle phalanx;
wherein all of the proximal, middle, and distal phalanges are covered by a flexible thermoplastic material, and wherein each of the distal phalanx includes an internal cavity having at least one sensor positioned within the internal cavity.

12. The prosthetic arm system of claim 9, where the forearm section is connected to an elbow section, which is further connected to an upper arm extension.

13. The prosthetic arm system of claim 9, further including a switch which permits for manual opening and closing of the hand.

14. The prosthetic arm system of claim 9 further including a system for controlling the prosthetic arm, the system comprising:
at least one non-transitory computer readable storage medium having computer program instructions stored thereon; and
at least one processor configured to execute the computer program instructions causing the processor to perform the following operations:
receive electroencephalography (EEG) signals from a user of a neurodetection device;
segment the received EEG signals by a collection duration to produce segmented EEG signals;
preprocess the segmented EEG signals by bandpass filtering, notch filtering, and transformer-based denoising to produce preprocessed EEG signals;
decode, using a machine-learning system, the preprocessed EEG signals to detect imagined spatial motion of the user, where the imagined spatial motion corresponds to desired motion commands of the prosthetic arm;
execute, using one or more of the plurality of actuators, detected imagined spatial motion of the user;
wherein the machine-learning system is a hybrid system including a first neural network and a second neural network, wherein the received EEG signals are input to the first neural network, wherein output of the first neural network is input to the second neural network, and wherein output of the second neural network is classification and identification of imagined spatial motion.

15. The prosthetic arm system of claim 9, further including a switch which permits for manual opening and closing of the hand.

16. The prosthetic arm system of claim 9 where the prosthetic arm is brain-controlled by use of:
a neurodetection device configured to acquire electroencephalography (EEG) signals from a user;
a preprocessing module configured to filter and denoise the EEG signals;
a hybrid neural network decoder configured to classify neural intent from the EEG signals; a control unit housed in the prosthetic arm and configured to activate one or more actuators of the prosthetic arm in response to the classified neural intent.

17. A system for controlling a prosthetic arm including a plurality of actuators, the system comprising:
at least one non-transitory computer readable storage medium having computer program instructions stored thereon; and
at least one processor configured to execute the computer program instructions causing the processor to perform the following operations:
receive electroencephalography (EEG) signals from a user of a neurodetection device;
segment the received EEG signals by a collection duration to produce segmented EEG signals;
preprocess the segmented EEG signals by bandpass filtering, notch filtering, and transformer denoising to produce preprocessed EEG signals;
decode, using a machine-learning system, the preprocessed EEG signals to detect imagined spatial motion of the user, where the imagined spatial motion corresponds to desired motion commands of the prosthetic arm;
execute, using one or more of the plurality of actuators, detected imagined spatial motion of the user;
wherein the machine-learning system is a hybrid system including a first neural network and a second neural network, wherein the received EEG signals are input to the first neural network, wherein output of the first neural network is input to the second neural network, and wherein output of the second neural network is classification and identification of imagined spatial motion.

18. The system of claim 15, wherein the neurodetection device includes a plurality of EEG signal-detecting electrodes worn on the user's head.
